(19) United States
(12) Patent Application Publication
(10) Pub. No.: US 2024/0052322 A1
(43) Pub. Date: Feb. 15, 2024

(54) HILIC UPLC-MS Method For Separating and Analyzing Intact Adeno-Associated Virus Capsid Proteins

(71) Applicant: Pfizer Inc., New York, NY (US)

(72) Inventors: Mark Thomas Chipley, O'Fallon, MO (US); Thomas Wesley Powers, Wildwood, MO (US)

(73) Assignee: Pfizer Inc., New York, NY (US)

(21) Appl. No.: 18/257,215

(22) PCT Filed: Dec. 13, 2021
(86) PCT No.: PCT/IB2021/061642

§ 371 Date: Jun. 13, 2023

Related U.S. Application Data
(60) Provisional application No. 63/125,689, filed on Dec. 15, 2020.

(51) Int. Cl. C12N 7/02; B01D 15/30; C12N 15/86

(52) U.S. Cl. CPC C12N 7/02; B01D 15/305; C12N 15/86; C12N 2750/14151; C12N 2750/14143

(57) ABSTRACT

Chromatographic method for separation of AAV capsid proteins using hydrophilic-interaction chromatography (HILIC). The method provides the ability to quantify capsid protein ratio and to separate capsid proteins to the extent that low level post-translational modifications (PTMs) can be detected by mass spectrometry.

### STATEMENT REGARDING FEDERALLY FUNDED RESEARCH

None.

### FIELD OF THE INVENTION

The field of the invention concerns molecular biology, cell biology, gene therapy, gene expression, and/or medicine. In particular aspects the invention relates to chromatographic procedures for separating adeno-associated virus (AAV) viral capsid proteins and then quantitatively and qualitatively evaluating and/or characterizing them, including by serotype.

### BACKGROUND OF THE INVENTION

Adeno-associated virus (AAV) is a leading gene therapy delivery platform being used for human therapeutics. Currently, there are three approved gene therapies utilizing AAV. Alipogene tiparvovec (Glybera) was approved in 2012 in the European Union. In the United States, voretigene neparvovec (Luxturna) was approved in 2017 and onasemnogene abeparvovec-xio (ZOLGENSMA) was approved in 2019. There are more than 100 AAV-based clinical trials ongoing, and there exists a need to develop new analytical methods that can be used to properly characterize and identify viral capsids essential for use in gene therapy. See, e.g., Wang, et al. (2018). Nature Reviews Drug Discovery, 18(5), 358-378; Kohn, D. B. (2018). Current Opinion in Biotechnology, 60, 39-45; Rodrigues, et al. (2018). Pharmaceutical Research, 36(2), 29; Smalley, E. (2017). Nature Biotechnology, 35(11), 998-999; and Strimvelis, et al. (2019). Nature Biotechnology, 37(7), 697. AAV is composed of an icosahedral capsid that encases an approximately 4.7 kb single stranded DNA genome. The wild-type AAV genome contains two genes encoding capsid and replication proteins. Multiple start codons and alternate mRNA splicing give rise to three overlapping capsid proteins (VP1, VP2, and VP3). Capsid proteins differ only at their N-termini and are present in a 1:1:8 ratio. VP1 contains a unique N-terminal region of about 137 amino acids, followed by a 65 amino acid region, common to VP1 and VP2. The VP3 protein contains approximately 534 amino acids (depending on the serotype), common to all 3 capsid proteins. Therapeutic AAV vectors consist of the AAV protein capsid containing a therapeutic gene for targeted delivery and expression. See Agbandje-McKenna, et al. (2011). Methods in Molecular Biology, 807, 47-92; Dunbar, et al. (2018). Science, 359(6372), eaan4672; Lerch, et al. (2010). Virology, 403(1), 26-36 and Xie, et al. (2002). Proceedings of the National Academy of Sciences USA, 99(16), 10405-10410. The protein capsid is critical for therapeutic gene delivery because of its role in the viral cell entry pathway(s). Post-translational modifications (PTM) of AAV capsids are not yet fully understood; however, measuring and understanding capsid quality attributes is important to the future development of therapeutic AAV vectors.

Recently, it has been reported that deamidation of capsid residues can lower vector infectivity. Traditionally, reverse phase high performance chromatography (RP-HPLC) has been used to characterize deamidation and other PTMs in biologics. Additionally, successful intact and subunit RP-HPLC separations have been reported for large biopharmaceuticals, such as monoclonal antibodies, achieving separation for proteins ranging in size from 25-150 kDa. AAV capsid proteins range in size from 61-73 kDa. Typically, proteins of this size would be retained and separated by RP-HPLC. However, this is not sufficient to obtain suitable resolution of the capsid VP1, VP2, and VP3 proteins, and to effectively evaluate the unique complexity of AAV vectors. AAV capsid proteins are interlocked into a highly stable higher order structure, and denaturation is required before chromatographic analysis. However, disassembled capsid proteins tend to form oligomers that lead to aggregation and precipitation. Intact virial particles are thermally stable; temperatures as high as 89° C. are needed to denature aqueous solutions of AAV. For RP-H PLC, denaturing the capsids prior to injection (i.e., within the vial) can therefore lead to time-dependent sample loss, limiting the method robustness. In addition, with bio-process development in progress to improve production yields, it is desirable to have a scalable analytical method that can be performed on small sample volumes.

### SUMMARY OF THE INVENTION

Chromatographic methods for separating AAV capsid proteins using hydrophilic-interaction chromatography (HILIC) are described. The method was developed to quantify capsid protein ratio and to separate capsid proteins to the extent that low level post translational modifications (PTMs) can be detected by mass spectrometry. Benefits to this method include that the high organic strength of mobile phases used in HILIC can denature the capsid on-column, such as when the particle enters the flow path or when the particles are in contact with the HILIC column. This allows for direct injection of viral particles onto the column, that can mitigate any loss due to sample preparation. This also allows for use of a lower column temperature, as the high temperatures used for RP-HPLC are not required to denature and separate the capsid proteins. Moreover, use of relatively high column temperature might result in deamidation, oxidation, fragmentation, aggregation of one or more proteins in the column, and loss of signal due to lower column capacity and/or binding. Such method artifacts can be reduced using lower column temperature. Further, the water layer formed on the stationary phase can be removed and regenerated, which would lead to low carry-over of analytes between injections. Also, the method can accommodate low-volume injections to suit the needs of discovery and early development phases. Use of lower amounts of material is more cost effective and saves more material for patient treatment. In some embodiments, as little as 1 μL of sample provides sufficient material for characterization and quantification. It has been shown herein that as little as 1 μl of sample can be used with HILIC coupled with a 1 mm I.D. column and still allows for mass spectrometry analysis. The method can be used to rapidly screen for rAAV or AAV product quality during process and formulation development while consuming a minor amount of material. Methods are provided concerning separating an adeno-associated virus (AAV) viral capsid into its components and determining the heterogeneity of an adeno-associated virus (AAV) particle by using hydrophilic-interaction chromatography (HILIC), which can be coupled with mass spectrometry. In some embodiments, there are methods that can be used to separate particular AAV or recombinant adeno-associated virus (rAAV) capsid serotypes, such as AAV9 or an rAAV9, and determine the heterogeneity of the particular AAV or rAAV serotype by separating and characterizing its AAV or rAAV capsid components using HILIC UPLC-MS. In some embodiments, the method can separate VP1, VP2, VP3 and their modified forms for multiple serotypes. The method works for multiple serotypes and can serve as a platform method for intact capsid protein analysis.

An embodiment of the invention, E1, is a method for separating proteins of a capsid of an adeno-associated virus (AAV) or recombinant adeno-associated virus (rAAV) particle, the method comprising:
a) loading the AAV particle onto a hydrophilic interaction liquid chromatography (HILIC) column; and
b) eluting the HILIC column with a mobile phase comprising a 99.9 vol. % or more of a mixture of water, acetonitrile, and trifluoroacetic acid (TFA) to obtain an eluent, wherein the starting water concentration in the mobile phase is 25 to 30 vol. % and during elution water concentration of the mobile phase is increased at a rate 0.3 to 0.6 vol. % per minute, and wherein TFA concentration in the mobile phase is 10 mM to about 30 mM.

An embodiment of the invention, E2, is the method of embodiment E1, further comprising performing mass spectrometry on at least a portion of the eluent and determining masses of one or more proteins in the eluent by mass spectrometry.

An embodiment of the invention, E3, is the method of any one of embodiments E1 to E2, wherein the AAV or rAAV particle is loaded to the HILIC column by direct injection.

An embodiment of the invention, E4, is the method of any one of embodiments E1 to E3, wherein during elution the mobile phase has a column flow rate of 0.10 mL/min to 0.16 mL/min.

An embodiment of the invention, E5, is the method of any one of embodiments E1 to E4, wherein the step b) has a run time of 25 to 30 min.

An embodiment of the invention, E6, is the method of any one of embodiments E1 to E5, wherein the starting water concentration in the mobile phase is 27 to 29 volume percent.

An embodiment of the invention, E7, is the method of any one of embodiments E1 to E6, wherein TFA concentration in the mobile phase is 17 mM to 23 mM.

An embodiment of the invention, E8, is the method of any one of embodiments E1 to E7, wherein the water concentration of the mobile phase is increased at a rate of 0.35 to 0.45 volume percentage per minute.

An embodiment of the invention, E9, is the method of any one of embodiments E1 to E8, wherein the AAV or rAAV particle is loaded onto the HILIC column by direct injection of a neat sample of the AAV or rAAV particle.

An embodiment of the invention, E10, is the method of any one of embodiments E1 to E9, wherein the AAV or rAAV particle is loaded onto the HILIC column by direct injection of a sample of the AAV or rAAV particle in phosphate buffered saline (PBS).

An embodiment of the invention, E11, is the method of any one of embodiments E2 to E10, wherein the mass spectrometer is run in positive-ion mode with a detection range of 600 to 5000 m/z.

An embodiment of the invention, E12, is the method of any one of embodiments E2 to E11, wherein the mass spectrometry comprises a capillary voltage of about 4.5 kV.

An embodiment of the invention, E13, is the method of any one of embodiments E1 to E12, wherein the AAV particle serotype is selected from the group consisting of AAV1, AAV2, AAV3, AAV3A, AAV3B, AAV4, AAV5, AAV6, AAV7, AAV8, AAV9, AAV10, AAVrh10, AAVrh74, AAV12, AAV2i8, NP4, NP22, NP66, AAVDJ, AAVDJ/8, AAVDJ/9, AAVLK03, AAV1.1, AAV2.5, AAV6.1, AAV6.3.1, AAV9.45, RHM4-1 (SEQ ID NO:5 of WO 2015/013313), RHM15-1, RHM15-2, RHM15-3/RHM15-5, RHM15-4, RHM15-6, AAV hu.26, AAV1.1, AAV2.5, AAV6.1, AAV6.3.1, AAV9,45, AAV2i8, AAV29G, AAV2,8G9, AVV-LK03, AAV2-TT, AAV2-TT-S312N, AAV3B-S312N, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14, and AAVHSC15.

An embodiment of the invention, E14, is the method of any one of embodiments E1 to E13, wherein the starting water concentration in the mobile phase is about 28 volume percent, the water concentration of the mobile phase is increased at a rate about 0.4 volume percentage per minute, TFA concentration in the mobile phase is about 20 mM, and the mobile phase has a column flow rate of about 0.14 mL/min.

An embodiment of the invention, E15, is the method of any one of embodiments E1 to E14, wherein the HILIC column comprises a stationary phase comprising amide functional groups.

An embodiment of the invention, E16, is the method of embodiment E15, wherein the stationary phase of the HILIC column comprises ethylene bridged hybrid (BEH) amide.

An embodiment of the invention, E17, is the method of any one of embodiments E1 to E16, wherein HILIC is UltraPerformance HILIC.

An embodiment of the invention, E18, is the method of any one of embodiments E1 to E17, wherein the HILIC column temperature during elution of the mobile phase is 25° C. to 40° C.

An embodiment of the invention, E19, is the method of embodiment E18, wherein the HILIC column temperature during elution of the mobile phase is 28° C. to 32° C.

An embodiment of the invention, E20, is the method of any one of embodiments E1 to E19, wherein a load solution containing the AAV or rAAV particle is loaded onto the column at a volume equal to 0.1 to 5 vol. % of the column.

An embodiment of the invention, E21, is the method of embodiment E20, wherein the load solution is loaded onto the column at a volume equal to 0.1 to 1 vol. % of the column.

An embodiment of the invention, E22, is the method of any one of embodiments E20 to E21, wherein load solution comprises 5×10¹⁰ to 1×10¹² virus particle (vp)/ml.

An embodiment of the invention, E23, is the method of embodiment E22, wherein the load solution comprises 9×10¹⁰ to 5×10¹¹ vp/ml.

An embodiment of the invention, E24, is the method of any one of embodiments E1 to E23, wherein intact AAV or rAAV particle is loaded to the HILIC.

An embodiment of the invention, E25, is the method of embodiment E24, wherein the intact AAV or rAAV capsid encapsidates a nucleic acid sequence.

An embodiment of the invention, E26, is the method of any one of embodiments E1 to E25, wherein amounts of one or more proteins in the eluent is measured.

An embodiment of the invention, E27, is the method of any one of embodiments E2 to E26, wherein the one or more proteins is selected from VP1, VP2, VP3, one or more post translation modification (PTM)s of VP1, one or more PTMs of VP2, and one or more PTMs of VP3, or any combination thereof.

An embodiment of the invention, E28, is the method of embodiment E27, wherein PTMS of VP1, VP2, and/or VP3 comprises independently acetylation, phosphorylation, deamidation, and/or oxidation thereof, or any combination thereof.

An embodiment of the invention, E29, is the method of any one of embodiments E2 to E28, wherein masses and/or amounts of the one or more proteins is indicative of serotype of the AAV capsid.

An embodiment of the invention, E30, is the method of any one of embodiments E2 to E29, wherein masses and/or amounts of the one or more proteins is indicative of heterogeneity of the AAV capsid.

An embodiment of the invention, E31, is the method of embodiment E30, wherein heterogeneity comprises oxidized capsids, phosphorylated capsids, acetylated capsids, or truncated capsids, or any combination thereof.

An embodiment of the invention, E32, is the method of any one of embodiments E1 to E31, wherein the AAV particle is a AAV9 particle.

An embodiment of the invention, E33, is the method of any one of embodiments E1 to E32, wherein the rAAV particle is a rAAV9 particle.

An embodiment of the invention, E34, is the method of any one of embodiments E2 to E33, wherein the AAV particle is AAV9 or rAAV9, and the one or more proteins is selected from a) VP1, b) des-methionine and acetylated VP1, c) des-methionine, acetylated, and deamidated VP1, d) des-methionine, acetylated, and phosphorylated VP1, e) VP2, f) des-threonine VP2, g) des-threonine, and deamidated VP2, h) des-threonine, and oxidized VP2, i) des-threonine and phosphorylated VP2, j) VP3, k) des-methionine and acetylated VP3, l) des-methionine, acetylated, and deamidated VP3, and m) des-methionine, acetylated and phosphorylated VP3, or any combination thereof.

An embodiment of the invention, E35, is the method of embodiment E34, wherein the one or more proteins is selected from a) VP1, b) des-methionine and acetylated VP1, c) des-methionine, acetylated, and deamidated VP1, and d) des-methionine, acetylated, and phosphorylated VP1, or any combination thereof.

An embodiment of the invention, E36, is the method of embodiment E35, wherein two or more proteins are selected from a) VP1, b) des-methionine and acetylated VP1, c) des-methionine, acetylated, and deamidated VP1, and d) des-methionine, acetylated, and phosphorylated VP1, or any combination thereof.

An embodiment of the invention, E37, is the method of embodiment E34, wherein the one or more proteins is selected from a) VP2, b) des-threonine VP2, c) des-threonine, and deamidated VP2, d) des-threonine, and oxidized VP2, and e) des-threonine and phosphorylated VP2, or any combination thereof.

An embodiment of the invention, E38, is the method of embodiment E37, wherein two or more proteins are selected from a) VP2, b) des-threonine VP2, c) des-threonine, and deamidated VP2, d) des-threonine, and oxidized VP2, and e) des-threonine and phosphorylated VP2, or any combination thereof.

An embodiment of the invention, E39, is the method of embodiment E34, wherein the one or more proteins is selected from a) VP3, b) des-methionine and acetylated VP3, c) des-methionine, acetylated, and deamidated VP3, and d) des-methionine, acetylated, and phosphorylated VP3, or any combination thereof.

An embodiment of the invention, E40, is the method of embodiment E39, wherein the two or more proteins are selected from a) VP3, b) des-methionine and acetylated VP3, c) des-methionine, acetylated, and deamidated VP3, and d) des-methionine, acetylated and phosphorylated VP3, or any combination thereof.

An embodiment of the invention, E41, is the method of any one of embodiments E1 to E31, wherein the AAV particle is a AAV1 particle.

An embodiment of the invention, E42, is the method of any one of embodiments E2 to E31, wherein the AAV particle is AAV1, and the one or more proteins is selected from a) VP1, b) des-methionine and acetylated VP1, c) des-methionine, acetylated, and deamidated VP1, d) des-methionine, acetylated, and phosphorylated VP1, e) VP2, f) des-threonine VP2, g) des-threonine and phosphorylated VP2, h) VP3, i) des-methionine and acetylated VP3, j) des-methionine, acetylated, and oxidized VP3, and k) des-methionine, acetylated, and phosphorylated VP3, or any combination thereof.

An embodiment of the invention, E43, is the method of embodiment E42, wherein the one or more proteins is selected from a) VP1, b) des-methionine and acetylated VP1, c) des-methionine, acetylated, and deamidated VP1, and d) des-methionine, acetylated, and phosphorylated VP1, or any combination thereof.

An embodiment of the invention, E44, is the method of embodiment E43, wherein two or more proteins are selected from a) VP1, b) des-methionine and acetylated VP1, c) des-methionine, acetylated, and deamidated VP1, and d) des-methionine, acetylated, and phosphorylated VP1, or any combination thereof.

An embodiment of the invention, E45, is the method of embodiment E42, wherein the one or more proteins is selected from a) VP2, b) des-threonine VP2 and c) des-threonine and phosphorylated VP2.

An embodiment of the invention, E46, is the method of embodiment E45, wherein two or more proteins are selected from a) VP2, b) des-threonine VP2, and c) des-threonine and phosphorylated VP2, or any combination thereof.

An embodiment of the invention, E47, is the method of embodiment E42, wherein the one or more proteins is selected a) VP3, b) des-methionine and acetylated VP3, c) des-methionine, acetylated, and oxidized VP3, and d) des-methionine, acetylated, and phosphorylated VP3, or any combination thereof.

An embodiment of the invention, E48, is the method of embodiment E47, wherein two or more proteins are selected from a) VP3, b) des-methionine and acetylated VP3, c) des-methionine, acetylated, and oxidized VP3, and d) des-methionine, acetylated, and phosphorylated VP3, or any combination thereof.

An embodiment of the invention, E49, is the method of any one of embodiments E1 to E31, wherein the AAV particle is a AAV2 particle.

An embodiment of the invention, E50, is the method of embodiment E2 to E31, wherein the AAV particle is AAV2, and the one or more proteins is selected from a) VP1, b) des-methionine and acetylated VP1, c) des-methionine, acetylated, and oxidized VP1, d) des-methionine, acetylated, and phosphorylated VP1, e) VP2, f) des-threonine VP2, g) des-threonine and phosphorylated VP2, h) VP3, i) des-methionine and acetylated VP3, j) des-methionine, acetylated, and oxidized VP3, and k) des-methionine, acetylated, and phosphorylated VP3, or any combination thereof.

An embodiment of the invention, E51, is the method of embodiment E50, wherein the one or more proteins is selected from a) VP1, b) des-methionine and acetylated VP1, c) des-methionine, acetylated, and oxidized VP1, and d) des-methionine, acetylated, and phosphorylated VP1, or any combination thereof.

An embodiment of the invention, E52, is the method of embodiment E51, wherein two or more proteins are selected from a) VP1, b) des-methionine and acetylated VP1, c) des-methionine, acetylated, and oxidized VP1 and d) des-methionine, acetylated, and phosphorylated VP1, or any combination thereof.

An embodiment of the invention, E53, is the method of embodiment E50, wherein the one or more proteins is selected from a) VP2, b) des-threonine VP2, and c) des-threonine and phosphorylated VP2, or any combination thereof.

An embodiment of the invention, E54, is the method of embodiment E53, wherein two or more proteins are selected from a) VP2, b) des-threonine VP2, and c) des-threonine and phosphorylated VP2, or any combination thereof.

An embodiment of the invention, E55, is the method of embodiment E50, wherein the one or more proteins is selected a) VP3, b) des-methionine and acetylated VP3, c) des-methionine, acetylated, and oxidized VP3, and d) des-methionine, acetylated, and phosphorylated VP3, or any combination thereof.

An embodiment of the invention, E56, is the method of embodiment E55, wherein two or more proteins are selected from a) VP3, b) des-methionine and acetylated VP3, c) des-methionine, acetylated, and oxidized VP3, and d) des-methionine, acetylated, and phosphorylated VP3, or any combination thereof.

An embodiment of the invention, E57, is the method of any one of embodiments E1 to E31, wherein the AAV particle is a AAV5 particle.

An embodiment of the invention, E58, is the method of any one of embodiments E2 to E31, wherein the AAV particle is AAV5, and the one or more proteins is selected from a) VP1, b) des-methionine and acetylated VP1, c) VP2, d) des-threonine VP2, e) des-threonine, and acetylated VP2, f) acetylated N-terminal methionine VP2, g) VP3, h) des-methionine and acetylated VP3, and i) des-methionine, acetylated, and deamidated VP3, or any combination thereof.

An embodiment of the invention, E59, is the method of embodiment E58, wherein the one or more proteins is selected from a) VP1 and b) des-methionine and acetylated VP1.

An embodiment of the invention, E60, is the method of embodiment E59, wherein a) VP1 and b) des-methionine and acetylated VP1 is selected.

An embodiment of the invention, E61, is the method of embodiment E58, wherein the one or more proteins is selected from a) VP2, b) des-threonine VP2, c) des-threonine, and acetylated VP2, and d) acetylated N-terminal methionine VP2, or any combination thereof.

An embodiment of the invention, E62, is the method of embodiment E61, wherein two or more proteins are selected from a) VP2, b) des-threonine VP2, c) des-threonine, and acetylated VP2 and d) acetylated N-terminal methionine VP2, or any combination thereof.

An embodiment of the invention, E63, is the method of embodiment E58, one or more proteins is selected a) VP3, b) des-methionine and acetylated VP3, and c) des-methionine, acetylated, and deamidated VP3.

An embodiment of the invention, E64, is the method of embodiment E63, wherein two or more proteins are selected from a) VP3, b) des-methionine and acetylated VP3, and c) des-methionine, acetylated, and deamidated VP3, or any combination thereof.

An embodiment of the invention, E65, is the method of any one of embodiments E1 to E31, wherein the AAV particle is a AAV6 particle.

An embodiment of the invention, E66, is the method of any one of embodiments E2 to E31, wherein the AAV particle is AAV6, and the one or more proteins is selected from a) VP1, b) des-methionine and acetylated VP1, c) des-methionine, acetylated, and oxidized VP1, d) des-methionine, acetylated, and phosphorylated VP1, e) VP2, f) des-threonine VP2, g) des-threonine and phosphorylated VP2, h) VP3, i) des-methionine and acetylated VP3, j) des-methionine, acetylated, and oxidized VP3 and k) des-methionine, acetylated, and phosphorylated VP3, or any combination thereof.

An embodiment of the invention, E67, is the method of embodiment E66, wherein the one or more proteins is selected from a) VP1, b) des-methionine and acetylated VP1, and c) des-methionine, acetylated, and phosphorylated VP1, or any combination thereof.

An embodiment of the invention, E68, is the method of embodiment E67, wherein two or more proteins are selected from a) VP1, b) des-methionine and acetylated VP1, and c) des-methionine, acetylated, and phosphorylated VP1, or any combination thereof.

An embodiment of the invention, E69, is the method of embodiment E66, wherein the one or more proteins is selected from a) VP2, b) des-threonine VP2, and c) des-threonine and phosphorylated VP2, or any combination thereof.

An embodiment of the invention, E70, is the method of embodiment E69, wherein two or more proteins are selected from a) VP2, b) des-threonine VP2, and c) des-threonine and phosphorylated VP2, or any combination thereof.

An embodiment of the invention, E71, is the method of embodiment E66, wherein the one or more proteins is selected a) VP3, b) des-methionine and acetylated VP3, c) des-methionine, acetylated, and oxidized VP3, and d) des-methionine, acetylated, and phosphorylated VP3, or any combination thereof.

An embodiment of the invention, E72, is the method of embodiment E71, wherein two or more proteins are selected from a) VP3, b) des-methionine and acetylated VP3, c) des-methionine, acetylated, and oxidized VP3, and d) des-methionine, acetylated, and phosphorylated VP3, or any combination thereof.

An embodiment of the invention, E73, is the method of any one of embodiments E1 to E31, wherein the AAV particle is a AAV8 particle.

An embodiment of the invention, E74, is the method of embodiment E2 to E31, wherein the AAV particle is AAV8, and the one or more proteins is selected from a) VP1, b) des-methionine and acetylated VP1, c) des-methionine, acetylated, and oxidized VP1, d) des-methionine, acetylated, and phosphorylated VP1, e) VP2, f) des-threonine VP2, g) des-threonine and phosphorylated VP2, h) des-threonine and phosphorylated VP2, and d) des-threonine, and bisphosphorylated VP2, i) VP3, j) des-methionine and acetylated VP3, k) des-methionine, acetylated, and oxidized VP3, l) des-methionine, and m) des-methionine, acetylated, and phosphorylated VP3, or any combination thereof.

An embodiment of the invention, E75, is the method of embodiment E74, wherein the one or more proteins is selected from a) VP1, b) des-methionine and acetylated VP1, and c) des-methionine, acetylated, and phosphorylated VP1, or any combination thereof.

An embodiment of the invention, E76, is the method of embodiment E75, wherein two or more proteins are selected from a) VP1, b) des-methionine and acetylated VP1, and c) des-methionine, acetylated, and phosphorylated VP1, or any combination thereof.

An embodiment of the invention, E77, is the method of embodiment E74, wherein the one or more proteins is selected from a) VP2, b) des-threonine VP2, c) des-threonine and phosphorylated VP2, and d) des-threonine, and bisphosphorylated VP2, or any combination thereof.

An embodiment of the invention, E78, is the method of embodiment E77, wherein two or more proteins are selected from a) VP2, b) des-threonine VP2, c) des-threonine and phosphorylated VP2, and d) des-threonine, and bisphosphorylated VP2, or any combination thereof.

An embodiment of the invention, E79, is the method of embodiment E74, wherein the one or more proteins is selected a) VP3, b) des-methionine and acetylated VP3, c) des-methionine, acetylated, and oxidized VP3, d) des-methionine, and e) des-methionine, acetylated, and phosphorylated VP3, or any combination thereof.

An embodiment of the invention, E80, is the method of embodiment E79, wherein two or more proteins are selected from a) VP3, b) des-methionine and acetylated VP3, c) des-methionine, acetylated, and oxidized VP3, d) des-methionine, and e) des-methionine, acetylated, and phosphorylated VP3, or any combination thereof.

An embodiment of the invention, E81, is the method of any one of embodiments E2 to 80, one or more capsid proteins determined with mass spectrometry is compared with reference masses to the one or more capsid proteins.

An embodiment of the invention, E82, is the method of embodiment E24, wherein the intact AAV or rAAV capsid is a null capsid.

An embodiment of the invention, E83, is a method for separating proteins of a capsid of an adeno-associated virus (AAV) or recombinant adeno-associated virus (rAAV) particle, the method comprising:
a) loading the AAV particle onto a hydrophilic interaction liquid chromatography (HILIC) column;
b) eluting the HILIC column with a mobile phase comprising a 99.9 vol. % or more of a mixture of water, acetonitrile, and trifluoroacetic acid (TFA) to obtain an eluent, wherein the starting water concentration in the mobile phase is 25 to 30 vol. % and during elution water concentration of the mobile phase is increased at a rate 0.3 to 0.6 vol. % per minute, and wherein TFA concentration in the mobile phase is 10 mM to about 30 mM;
c) performing mass spectrometry on at least a portion of the eluent and determining masses of one or more proteins in the eluant by mass spectrometry; and
d) comparing the masses of the one or more capsid proteins determined in step c) with their expected theoretical mass.

An embodiment of the invention, E84, is the method of embodiment E83, wherein the AAV or rAAV particle is loaded to the HILIC column by direct injection.

An embodiment of the invention, E85, is the method of any one of embodiments E83 to E84, wherein during elution the mobile phase has a column flow rate of 0.10 mL/min to 0.16 mL/min.

An embodiment of the invention, E86, is the method of any one of embodiments E83 to E85, wherein the step b) has a run time of 25 to 30 min.

An embodiment of the invention, E87, is the method of any one of embodiments E83 to E86, wherein the starting water concentration in the mobile phase is 27 to 29 volume percent.

An embodiment of the invention, E88, is the method of any one of embodiments E83 to E87, wherein TFA concentration in the mobile phase is 17 mM to 23 mM.

An embodiment of the invention, E89, is the method of any one of embodiments E83 to E88, wherein the water concentration of the mobile phase is increased at a rate 0.35 to 0.45 volume percentage per minute.

An embodiment of the invention, E90, is the method of any one of embodiments E83 to E89, wherein the AAV or rAAV particle is loaded onto the HILIC column by direct injection of a neat sample of the AAV or rAAV particle.

An embodiment of the invention, E91, is the method of any one of embodiments E83 to E90, wherein the AAV or rAAV particle is loaded onto the HILIC column by direct injection of a sample of the AAV or rAAV particle in phosphate buffered saline (PBS).

An embodiment of the invention, E92, is the method of any one of embodiments E83 to E91, wherein the mass spectrometer is run in positive-ion mode with a detection range of 600-5000 m/z.

An embodiment of the invention, E93, is the method of any one of embodiments E83 to E92, wherein the mass spectrometry comprises a capillary voltage of about 4.5 kV.

An embodiment of the invention, E94, is the method of any one of embodiments E83 to E93, wherein the AAV particle serotype is selected from the group consisting of AAV1, AAV2, AAV3, AAV3A, AAV3B, AAV4, AAV5, AAV6, AAV7, AAV8, AAV9, AAV10, AAVrh10, AAVrh74, AAV12, AAV2i8, NP4, NP22, NP66, AAVDJ, AAVDJ/8, AAVDJ/9, AAVLK03, AAV1.1, AAV2.5, AAV6.1, AAV6.3.1, AAV9.45, RHM4-1 (SEQ ID NO:5 of WO 2015/013313), RHM15-1, RHM15-2, RHM15-3/RHM15-5, RHM15-4, RHM15-6, AAV hu.26, AAV1.1, AAV2.5, AAV6.1, AAV6.3.1, AAV9,45, AAV2i8, AAV29G, AAV2,8G9, AVV-LK03, AAV2-TT, AAV2-TT-S312N, AAV3B-S312N, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14, and AAVHSC15.

An embodiment of the invention, E95, is the method of any one of embodiments E83 to E94, wherein the starting water concentration in the mobile phase is about 28 volume percent, the water concentration of the mobile phase is increased at a rate about 0.4 volume percentage per minute, TFA concentration in the mobile phase is about 20 mM, the mobile phase has a column flow rate of about 0.14 mL/min, or combinations thereof.

An embodiment of the invention, E96, is the method of any one of embodiments E83 to E95, wherein the HILIC column comprises a stationary phase comprising amide functional groups.

An embodiment of the invention, E97, is the method of embodiment E96, wherein the stationary phase of the HILIC column comprises ethylene bridged hybrid (BEH) amide.

An embodiment of the invention, E98, is the method of any one of embodiments E83 to E97, wherein HILIC is UltraPerformance HILIC.

An embodiment of the invention, E99, is the method of any one of embodiments E83 to E98, wherein the HILIC column temperature during elution of the mobile phase is 25° C. to 40° C.

An embodiment of the invention, E100, is the method of embodiment E99, wherein the HILIC column temperature during elution of the mobile phase is 28° C. to 32° C.

An embodiment of the invention, E101, is the method of any one of embodiments E83 to E100, wherein a load solution containing the AAV or rAAV particle is loaded onto the column at a volume equal to 0.1 to 5 vol. % of the column.

An embodiment of the invention, E102, is the method of embodiment E101, wherein the load solution is loaded onto the column at a volume equal to 0.1 to 1 vol. % of the column.

An embodiment of the invention, E103, is the method of any one of embodiments E101 to E102, wherein load solution comprises 5×10¹⁰ to 1×10¹² virus particle (vp)/ml.

An embodiment of the invention, E104, is the method of embodiment E103, wherein the load solution comprises 9×10¹⁰ to 5×10¹¹ vp/ml.

An embodiment of the invention, E105, is the method of any one of embodiments E83 to E104, wherein intact AAV or rAAV particle is loaded to the HILIC.

An embodiment of the invention, E106, is the method of any one of embodiments E83 to E105, wherein the intact AAV or rAAV capsid encapsidates a nucleic acid sequence.

An embodiment of the invention, E107, is the method of any one of embodiments E83 to E106, wherein amounts of one or more proteins in the eluent is measured.

An embodiment of the invention, E108, is the method of any one of embodiments E83 to E107, wherein the one or more proteins is selected from VP1, VP2, VP3, one or more post translation modification (PTM)s of VP1, one or more PTMs of VP2, and one or more PTMs of VP3, or any combination thereof.

An embodiment of the invention, E109, is the method of embodiment E108, wherein PTMS of VP1, VP2, and/or VP3 comprises independently acetylation, phosphorylation, deamidation, and/or oxidation thereof, or any combination thereof.

An embodiment of the invention, E110, is the method of any one of embodiments E83 to E109, wherein masses and/or amounts of the one or more proteins is indicative of serotype of the AAV capsid.

An embodiment of the invention, E111, is the method of any one of embodiments E83 to E110, wherein masses and/or amounts of the one or more proteins is indicative of heterogeneity of the AAV capsid.

An embodiment of the invention, E112, is the method of embodiment E111, wherein heterogeneity comprises oxidized capsids, phosphorylated capsids, acetylated capsids, or truncated capsids, or any combination thereof.

An embodiment of the invention, E113, is the method of any one of embodiments E83 to E112, wherein the AAV particle is a AAV9 particle.

An embodiment of the invention, E114, is the method of any one of embodiments E83 to E112, wherein the rAAV particle is a rAAV9 particle.

An embodiment of the invention, E115, is the method of any one of embodiments E83 to E114, wherein the AAV particle is AAV9 or rAAV9, and the one or more proteins is selected from a) VP1, b) des-methionine and acetylated VP1, c) des-methionine, acetylated, and deamidated VP1, d) des-methionine, acetylated, and phosphorylated VP1, e) VP2, f) des-threonine VP2, g) des-threonine, and deamidated VP2, h) des-threonine, and oxidized VP2, i) des-threonine and phosphorylated VP2, j) VP3, k) des-methionine and acetylated VP3, l) des-methionine, acetylated, and deamidated VP3, and m) des-methionine, acetylated and phosphorylated VP3, or any combination thereof.

An embodiment of the invention, E116, is the method embodiment E115, wherein the one or more proteins is selected from a) VP1, b) des-methionine and acetylated VP1, c) des-methionine, acetylated, and deamidated VP1, and d) des-methionine, acetylated, and phosphorylated VP1, or any combination thereof.

An embodiment of the invention, E117, is the method embodiment E116, wherein two or more proteins are selected from a) VP1, b) des-methionine and acetylated VP1, c) des-methionine, acetylated, and deamidated VP1, and d) des-methionine, acetylated, and phosphorylated VP1, or any combination thereof.

An embodiment of the invention, E118, is the method embodiment E115, wherein the one or more proteins is selected from a) VP2, b) des-threonine VP2, c) des-threonine, and deamidated VP2, d) des-threonine, and oxidized VP2, and e) des-threonine and phosphorylated VP2, or any combination thereof.

An embodiment of the invention, E119, is the method embodiment E118, wherein two or more proteins are selected from a) VP2, b) des-threonine VP2, c) des-threonine, and deamidated VP2, d) des-threonine, and oxidized VP2, and e) des-threonine and phosphorylated VP2, or any combination thereof.

An embodiment of the invention, E120, is the method embodiment E115, wherein the one or more proteins is selected from a) VP3, b) des-methionine and acetylated VP3, c) des-methionine, acetylated, and deamidated VP3, and d) des-methionine, acetylated, and phosphorylated VP3, or any combination thereof.

An embodiment of the invention, E121, is the method embodiment E120, wherein the two or more proteins are selected from a) VP3, b) des-methionine and acetylated VP3, c) des-methionine, acetylated, and deamidated VP3, and d) des-methionine, acetylated and phosphorylated VP3, or any combination thereof.

An embodiment of the invention, E122, is the method of any one of embodiments E83 to E112, wherein the AAV particle is a AAV1 particle.

An embodiment of the invention, E123, is the method of any one of embodiments E83 to E112, wherein the AAV particle is AAV1, and the one or more proteins is selected from a) VP1, b) des-methionine and acetylated VP1, c) des-methionine, acetylated, and deamidated VP1, d) des-methionine, acetylated, and phosphorylated VP1, e) VP2, f) des-threonine VP2, g) des-threonine and phosphorylated VP2, h) VP3, i) des-methionine and acetylated VP3, j) des-methionine, acetylated, and oxidized VP3, and k) des-methionine, acetylated, and phosphorylated VP3, or any combination thereof.

An embodiment of the invention, E124, is the method of embodiment E123, wherein the one or more proteins is selected from a) VP1, b) des-methionine and acetylated VP1, c) des-methionine, acetylated, and deamidated VP1, and d) des-methionine, acetylated, and phosphorylated VP1, or any combination thereof.

An embodiment of the invention, E125, is the method of embodiment E124, wherein two or more proteins are selected from a) VP1, b) des-methionine and acetylated VP1, c) des-methionine, acetylated, and deamidated VP1, and d) des-methionine, acetylated, and phosphorylated VP1, or any combination thereof.

An embodiment of the invention, E126, is the method of embodiment E123, wherein the one or more proteins is selected from a) VP2, b) des-threonine VP2 and c) des-threonine and phosphorylated VP2.

An embodiment of the invention, E127, is the method of embodiment E126, wherein two or more proteins are selected from a) VP2, b) des-threonine VP2 and c) des-threonine and phosphorylated VP2, or any combination thereof.

An embodiment of the invention, E128, is the method of embodiment E123, wherein the one or more proteins is selected a) VP3, b) des-methionine and acetylated VP3, c) des-methionine, acetylated, and oxidized VP3, and d) des-methionine, acetylated, and phosphorylated VP3, or any combination thereof.

An embodiment of the invention, E129, is the method of embodiment E128, wherein two or more proteins are selected from a) VP3, b) des-methionine and acetylated VP3, c) des-methionine, acetylated, and oxidized VP3 and d) des-methionine, acetylated, and phosphorylated VP3, or any combination thereof.

An embodiment of the invention, E130, is the method of any one of embodiments E83 to E112, wherein the AAV particle is a AAV2 particle.

An embodiment of the invention, E131, is the method of any one of embodiments E83 to E112, wherein the AAV particle is AAV2, and the one or more proteins is selected from a) VP1, b) des-methionine and acetylated VP1, c) des-methionine, acetylated, and oxidized VP1, d) des-methionine, acetylated, and phosphorylated VP1, e) VP2, f) des-threonine VP2, g) des-threonine and phosphorylated VP2, h) VP3, i) des-methionine and acetylated VP3, j) des-methionine, acetylated, and oxidized VP3, and k) des-methionine, acetylated, and phosphorylated VP3, or any combination thereof.

An embodiment of the invention, E132, is the method of embodiment E131, wherein the one or more proteins is selected from a) VP1, b) des-methionine and acetylated VP1, c) des-methionine, acetylated, and oxidized VP1 and d) des-methionine, acetylated, and phosphorylated VP1, or any combination thereof.

An embodiment of the invention, E133, is the method of embodiment E132, wherein two or more proteins are selected from a) VP1, b) des-methionine and acetylated VP1, c) des-methionine, acetylated, and oxidized VP1 and d) des-methionine, acetylated, and phosphorylated VP1, or any combination thereof.

An embodiment of the invention, E134, is the method of embodiment E131, wherein the one or more proteins is selected from a) VP2, b) des-threonine VP2 and c) des-threonine and phosphorylated VP2, or any combination thereof.

An embodiment of the invention, E135, is the method of embodiment E134, wherein two or more proteins are selected from a) VP2, b) des-threonine VP2 and c) des-threonine and phosphorylated VP2, or any combination thereof.

An embodiment of the invention, E136, is the method of embodiment E131, wherein the one or more proteins is selected a) VP3, b) des-methionine and acetylated VP3, c) des-methionine, acetylated, and oxidized VP3 and d) des-methionine, acetylated, and phosphorylated VP3, or any combination thereof.

An embodiment of the invention, E137, is the method of embodiment E136, wherein two or more proteins are selected from a) VP3, b) des-methionine and acetylated VP3, c) des-methionine, acetylated, and oxidized VP3 and d) des-methionine, acetylated, and phosphorylated VP3, or any combination thereof.

An embodiment of the invention, E138, is the method of any one of embodiments E83 to E112, wherein the AAV particle is a AAV5 particle.

An embodiment of the invention, E139, is the method of any one of embodiments E83 to E112, wherein the AAV particle is AAV5, and the one or more proteins is selected from a) VP1, b) des-methionine and acetylated VP1, c) VP2, d) des-threonine VP2, e) des-threonine, and acetylated VP2, f) acetylated N-terminal methionine VP2, g) VP3, h) des-methionine and acetylated VP3, and i) des-methionine, acetylated, and deamidated VP3, or any combination thereof.

An embodiment of the invention, E140, is the method of embodiment E139, wherein the one or more proteins is selected from a) VP1 and b) des-methionine and acetylated VP1.

An embodiment of the invention, E141, is the method of embodiment E140, wherein a) VP1 and b) des-methionine and acetylated VP1 is selected.

An embodiment of the invention, E142, is the method of embodiment E139, wherein the one or more proteins is selected from a) VP2, b) des-threonine VP2, c) des-threonine, and acetylated VP2, and d) acetylated N-terminal methionine VP2, or any combination thereof.

An embodiment of the invention, E143, is the method of embodiment E142, wherein two or more proteins are selected from a) VP2, b) des-threonine VP2, c) des-threonine, and acetylated VP2 and d) acetylated N-terminal methionine VP2, or any combination thereof.

An embodiment of the invention, E144, is the method of embodiment E139, one or more proteins is selected a) VP3, b) des-methionine and acetylated VP3, and c) des-methionine, acetylated, and deamidated VP3.

An embodiment of the invention, E145, is the method of embodiment E144, wherein two or more proteins are selected from a) VP3, b) des-methionine and acetylated VP3, and c) des-methionine, acetylated, and deamidated VP3, or any combination thereof.

An embodiment of the invention, E146, is the method of any one of embodiments E83 to E112, wherein the AAV particle is a AAV6 particle.

An embodiment of the invention, E147, is the method of any one of embodiments E83 to E112, wherein the AAV particle is AAV6, and the one or more proteins is selected from a) VP1, b) des-methionine and acetylated VP1, c) des-methionine, acetylated, and oxidized VP1, d) des-methionine, acetylated, and phosphorylated VP1, e) VP2, f) des-threonine VP2, g) des-threonine and phosphorylated VP2, h) VP3, i) des-methionine and acetylated VP3, j) des-methionine, acetylated, and oxidized VP3 and k) des-methionine, acetylated, and phosphorylated VP3, or any combination thereof.

An embodiment of the invention, E148, is the method of embodiment E147, wherein the one or more proteins is selected from a) VP1, b) des-methionine and acetylated VP1, and c) des-methionine, acetylated, and phosphorylated VP1, or any combination thereof.

An embodiment of the invention, E149, is the method of embodiment E148, wherein two or more proteins are selected from a) VP1, b) des-methionine and acetylated VP1, and c) des-methionine, acetylated, and phosphorylated VP1, or any combination thereof.

An embodiment of the invention, E150, is the method of embodiment E147, wherein the one or more proteins is selected from a) VP2, b) des-threonine VP2, and c) des-threonine and phosphorylated VP2, or any combination thereof.

An embodiment of the invention, E151, is the method of embodiment E150, wherein two or more proteins are selected from a) VP2, b) des-threonine VP2, and c) des-threonine and phosphorylated VP2, or any combination thereof.

An embodiment of the invention, E152, is the method of embodiment E147, wherein the one or more proteins is selected a) VP3, b) des-methionine and acetylated VP3, c) des-methionine, acetylated, and oxidized VP3, and d) des-methionine, acetylated, and phosphorylated VP3, or any combination thereof.

An embodiment of the invention, E153, is the method of embodiment E152, wherein two or more proteins are selected from a) VP3, b) des-methionine and acetylated VP3, c) des-methionine, acetylated, and oxidized VP3, and d) des-methionine, acetylated, and phosphorylated VP3, or any combination thereof.

An embodiment of the invention, E154, is the method of any one of embodiments E83 to E112, wherein the AAV particle is a AAV8 particle.

An embodiment of the invention, E155, is the method of any one of embodiments E83 to E112, wherein the AAV particle is AAV8, and the one or more proteins is selected from a) VP1, b) des-methionine and acetylated VP1, c) des-methionine, acetylated, and oxidized VP1, d) des-methionine, acetylated, and phosphorylated VP1, e) VP2, f) des-threonine VP2, g) des-threonine and phosphorylated VP2, h) des-threonine and phosphorylated VP2, and d) des-threonine, and bisphosphorylated VP2, i) VP3, j) des-methionine and acetylated VP3, k) des-methionine, acetylated, and oxidized VP3, l) des-methionine, and m) des-methionine, acetylated, and phosphorylated VP3, or any combination thereof.

An embodiment of the invention, E156, is the method of embodiment E155, wherein the one or more proteins is selected from a) VP1, b) des-methionine and acetylated VP1, and c) des-methionine, acetylated, and phosphorylated VP1, or any combination thereof.

An embodiment of the invention, E157, is the method of embodiment E156, wherein two or more proteins are selected from a) VP1, b) des-methionine and acetylated VP1, and c) des-methionine, acetylated, and phosphorylated VP1, or any combination thereof.

An embodiment of the invention, E158, is the method of embodiment E155, wherein the one or more proteins is selected from a) VP2, b) des-threonine VP2, c) des-threonine and phosphorylated VP2, and d) des-threonine, and bisphosphorylated VP2, or any combination thereof.

An embodiment of the invention, E159, is the method of embodiment E158, wherein two or more proteins are selected from a) VP2, b) des-threonine VP2, c) des-threonine and phosphorylated VP2, and d) des-threonine, and bisphosphorylated VP2, or any combination thereof.

An embodiment of the invention, E160, is the method of embodiment E155, wherein the one or more proteins is selected a) VP3, b) des-methionine and acetylated VP3, c) des-methionine, acetylated, and oxidized VP3, d) des-methionine, and e) des-methionine, acetylated, and phosphorylated VP3, or any combination thereof.

An embodiment of the invention, E161, is the method of embodiment E160, wherein two or more proteins are selected from a) VP3, b) des-methionine and acetylated VP3, c) des-methionine, acetylated, and oxidized VP3, d) des-methionine, and e) des-methionine, acetylated, and phosphorylated VP3, or any combination thereof.

An embodiment of the invention, E162, is the method of embodiment E105, wherein the intact AAV or rAAV capsid is a null capsid.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or apparatus of the invention, and vice versa. Furthermore, compositions and apparatuses of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value. In some embodiments, the term “about” can be added to any numeral recited herein to the extent the numeral would have a standard deviation of error when measuring.

The use of the term “or” in the claims is used to mean “and/or” unless explicitly indicated to refer to alternatives only or the alternatives are mutually exclusive, although the disclosure supports a definition that refers to only alternatives and “and/or.” As used herein, the term “and/or” refers to and encompasses any and all possible combinations of one or more of the associated listed items, as well as the lack of combinations when interpreted in the alternative (“or”).

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a method that “comprises” a list of elements (e.g., components or features or steps) is not necessarily limited to only those elements (or components or features or steps), but may include other elements (or components or features or steps) not expressly listed or inherent to the method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”. Any embodiment described using the term “comprising” may be implemented in the context of the terms “consisting of” or “consisting essentially of”, and vice versa.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1: HILIC chromatogram of AAV9 capsid obtained using conditions as shown in Table 5. Peaks corresponding to VP1; VP2; and VP3 are marked with arrows.

FIG. 2: HILIC chromatogram of FIG. 1, zoomed in along the vertical axis. Peaks corresponding to VP1; VP2; VP3; VP1_{d}, VP 1ₚ (mixture of deamidated and phosphorylated VP1); VP2_{d}, VP2ₚ (mixture of deamidated and phosphorylated VP2); and VP3_{d}, VP3ₚ (mixture of deamidated and phosphorylated VP3) are marked with arrows.

FIG. 3: 2D density plot showing AAV9 capsid recovery as a function of starting water weight percent in the mobile phase and TFA concentrations in the mobile phase.

FIG. 4: 2D density plot showing AAV9 capsid retention as a function of starting water weight percent in the mobile phase and TFA concentrations in the mobile phase.

FIG. 5: 2D density plot showing difference of retention time of peaks corresponding to VP3 and VP1, as a function of starting water weight percent in the mobile phase and TFA concentrations in the mobile phase.

FIG. 6: 2D density plot showing difference of retention time of peaks corresponding to VP1 and VP2, as a function of starting water weight percent in the mobile phase and TFA concentrations in the mobile phase.

FIG. 7: 2D density plot showing peak capacity in HILIC chromatograms, as a function of elusion water gradients and column flow rates.

FIG. 8: 2D density plot showing differences in retention time of peaks corresponding to VP1, and VP1_{d}, VP1ₚ (mixture of deamidated and phosphorylated VP1), as a function of elusion gradients and column flow rates.

FIG. 9: HILIC chromatograms obtained using column temperatures of 25° C., 30° C., 35° C., and 40° C. Peaks corresponding to VP1; VP2; VP3; VP1_{d}, VP1ₚ (mixture of deamidated and phosphorylated VP1) are marked with arrows.

FIG. 10: Difference in retention time of the peaks corresponding to VP1_{d}, VP1ₚ (mixture of deamidated and phosphorylated VP1) and VP1 (filled circles); and retention time of the peaks corresponding to VP1_{d}, VP1ₚ and VP2 (filled squares), as a function of column temperatures.

FIG. 11: Five-point standard curve for addition of AAV9 viral capsid load. Average areas under the peak corresponding to VP1, VP2, and VP3 are plotted as a function of column load. Each load level was injected in duplicate.

FIG. 12: Ratio of area under the peaks corresponding to VP1, VP2, and VP3 as a function of column load.

FIG. 13: HILIC chromatogram of AAV1 capsid. Peaks corresponding to VP1; VP2ₚ (phosphorylated VP2); VP3; VP1_{d}, VP1ₚ, VP2 (mixture of VP2 and deamidated and phosphorylated VP1); and VP3_{Ox}, VP3ₚ (mixture of oxidized and phosphorylated VP3) are marked with arrows.

FIG. 14: HILIC chromatogram of AAV2 capsid. Peaks corresponding to VP1; VP2; VP2ₚ (phosphorylated VP2); VP3; VP1_{Ox}, VP1ₚ (mixture of oxidized and phosphorylated VP1); and VP3_{Ox}, VP3ₚ (mixture of oxidized and phosphorylated VP3) are marked with arrows.

FIG. 15: HILIC chromatogram of AAV5 capsid. Peaks corresponding to VP3; VP2; VP3d (deamidated VP3); and VP2, VP3 (mixture of VP2 and VP3) are marked with arrows.

FIG. 16: HILIC chromatogram of AAV6 capsid. Peaks corresponding to VP1; VP2ₚ (phosphorylated VP2); VP3; VP1ₚ, VP2 (mixture of VP2 and phosphorylated VP1); and VP3_{Ox}, VP3ₚ (mixture of oxidized and phosphorylated VP3) are marked with arrows.

FIG. 17: HILIC chromatogram of AAV8 capsid. Peaks corresponding to VP1; VP2ₚ (phosphorylated VP2); VP2_{p,p} (VP2 with two phosphorylations); VP3; VP1ₚ, VP2 (mixture of VP2 and phosphorylated VP1); and VP3_{w/o Acetylation} (VP3 missing an acetylation) are marked with arrows.

### DETAILED DESCRIPTION OF THE INVENTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Methods described herein can be used for separating and characterizing the component proteins of adeno-associated virus capsids, using hydrophilic-interaction chromatography coupled with mass spectrometry. In one aspect, the method can include, loading an AAV particle onto a hydrophilic interaction liquid chromatography (HILIC) column, eluting the column using a mobile phase to obtain an eluent containing proteins separated from the capsid of the AAV particle, and performing mass spectrometry on at least a portion of the eluent and determining masses of one or more proteins in the eluent by mass spectrometry.

I. AAV Particle

Viral proteins in Adeno-associated virus capsids can be separated and optionally characterized using methods described herein. AAV can be any natural AAV (e.g. exists in nature) or recombinant AAV (rAAV). AAV can be an AAV described herein. In some embodiments, one or more AAV described herein can be excluded. In some embodiments, a natural AAV particle can be loaded on to the HILIC column, and one or more viral proteins in the natural AAV capsid can be separated and optionally characterized using methods described herein. In some embodiments, a rAAV particle can be loaded on to the HILIC column, and one or more viral proteins in the rAAV capsid can be separated and optionally characterized using methods described herein.

The term “AAV particle” refers to AAV viral particle containing an AAV capsid. The capsid can be a null capsid, e.g., lack a vector genome or it can encapsidate a vector genome.

“Adeno-associated virus” and/or “AAV” refer to parvoviruses with a linear single-stranded DNA genome and variants thereof. The term covers all subtypes and both naturally occurring and recombinant forms, except where required otherwise. Parvoviruses, including AAV, are useful as gene therapy vectors as they can penetrate a cell and introduce a nucleic acid (e.g., transgene) into the nucleus. In some embodiments, the introduced nucleic acid (e.g., rAAV vector genome) forms circular concatemers that persist as episomes in the nucleus of transduced cells. In some embodiments, a transgene is inserted in specific sites in the host cell genome, for example at a site on human chromosome 19. Site-specific integration, as opposed to random integration, is believed to more likely result in a predictable long-term expression profile. The insertion site of AAV into the human genome is referred to as AAVS1. Once introduced into a cell, polypeptides encoded by the nucleic acid can be expressed by the cell. Because AAV is not associated with any pathogenic disease in humans, a nucleic acid delivered by AAV can be used to express a therapeutic polypeptide for the treatment of a disease, disorder and/or condition in a human subject.

Multiple serotypes of AAV exist in nature with at least fifteen wild type serotypes having been identified from humans thus far (i.e., AAV1-AAV15). Naturally occurring and variant serotypes are distinguished by a protein capsid that is serologically distinct from other AAV serotypes. AAV type 1 (AAV1), AAV type 2 (AAV2), AAV type 3 (AAV3) including AAV type 3A (AAV3A) and AAV type 3B (AAV3B), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 12 (AAV12), AAVrh10, AAVrh74 (see WO 2016/210170), avian AAV, bovine AAV, canine AAV, equine AAV, primate AAV, non-primate AAV, and ovine AAV, and recombinantly produced variants (e.g., capsid variants with insertions, deletions and substitutions, etc.), such as variants referred to as AAV type 2i8 (AAV2i8), NP4, NP22, N P66, AAVDJ, AAVDJ/8, AAVDJ/9, AAVLK03, RHM4-1, among many others. AAV variants isolated from human CD34+ cell include AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14 and AAVHSC15 (Smith, et al. (2014). Molecular Therapy, 22(9), 1625-1634).

“Primate AAV” refers to AAV that infect primates, “non-primate AAV” refers to AAV that infect non-primate animals, “bovine AAV” refers to AAV that infect bovine mammals, and so on. Serotype distinctiveness can be determined on the basis of the lack of cross-reactivity between antibodies to one AAV as compared to another AAV. Such cross-reactivity differences are usually due to differences in capsid protein sequences and antigenic determinants (e.g., due to VP1, VP2, and/or VP3 sequence differences of AAV serotypes). However, some naturally occurring AAV or engineered AAV mutants (e.g., recombinant AAV) may not exhibit serological difference with any of the currently known serotypes. These viruses may then be considered a subgroup of the corresponding type, or more simply a variant AAV. Thus, as used herein, the term “serotype” refers to both serologically distinct viruses, e.g., AAV, as well as viruses, that are not serologically distinct but that may be within a subgroup or a variant of a given serotype.

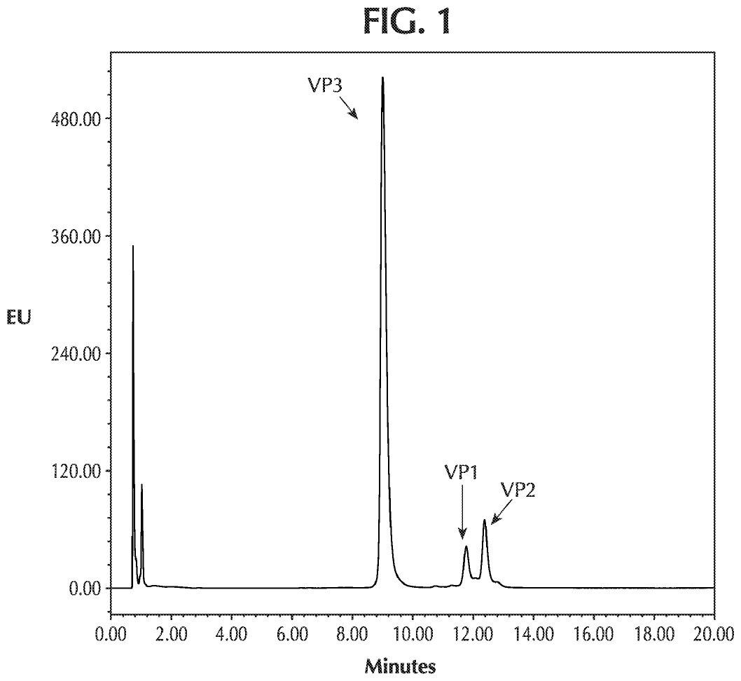

A non-limiting list and alignment of amino acid sequences of capsids of known AAV serotypes is provided by Marsic, et al. (2014). Molecular Therapy, 22(11), 1900-1909, especially at supplementary FIG. 1, which is hereby incorporated by reference. Non-limiting genomic sequences of various serotypes of AAV, as well as sequences of the native terminal repeats (ITRs), replication proteins, and capsid subunits are known in the art. Such sequences may be found in the literature or in public databases such as GenBank. See, e.g., GenBank Accession Numbers NC_002077 (AAV1), AF063497 (AAV1), NC_001401 (AAV2), AF043303 (AAV2), NC_001729 (AAV3), AF028705.1 (AAV3B), NC_001829 (AAV4), U89790 (AAV4), NC_006152 (AAV5), AF028704 (AAV6), AF513851 (AAV7), AF513852 (AAV8), NC_006261 (AAV8), AY530579 (AAV9), AY631965 (AAV10), AY631966 (AAV11), and DQ813647 (AAV12); the disclosures of which are incorporated by reference herein. See also, e.g., Srivistava, et al. (1983). Journal of Virology, 45, 555; Chiorini, et al. (1998). Journal of Virology, 71, 6823; Chiorini, et al. (1999). Journal of Virology, 73, 1309; Bantel-Schaal, et al. (1999). Journal of Virology, 73, 939; Xiao, et al. (1999). Journal of Virology, 73, 3994; Muramatsu, et al. (1996). Virology, 221, 208; Shade, et al. (1986). Journal of Virology, 58, 921; Gao, et al. (2002). Proceedings of the National Academy of Sciences USA, 99, 11854; Moris et al. (2004). Virology, 33, 375-383; International patent application publications WO 00/28061, WO 99/61601, WO 98/11244, WO 2013/063379, WO 2014/194132, and WO 2015/121501; U.S. Pat. Nos. 6,156,303; and 7,906,111, all of which are incorporated by reference. For illustrative purposes only, wild type AAV2 comprises a small (20-25 nm) icosahedral virus capsid of AAV composed of 60 capsid proteins made up of three different proteins (VP1, VP2, and VP3) with overlapping sequences. The proteins VP1 (735 aa; Genbank Accession No. AAC03780), VP2 (598 aa; Genbank Accession No. AAC03778), and VP3 (533 aa; Genbank Accession No. AAC03779) exist in a 1:1:10 ratio in the capsid. For AAVs, VP1 is the full-length protein and VP2 and VP3 are progressively shorter versions of VP1, typically with increasing truncation of the N-terminus relative to VP1; however, other truncations are known to occur (See for example Galibert, et al. (2018), PLoS ONE, 13(11), e0207414).

As discussed supra, a “recombinant adeno-associated virus” or “rAAV” is distinguished from a wild-type AAV by replacement of all or part of the viral genome with a non-native sequence or a viral capsid with a protein containing a non-native amino acid sequence or a viral capsid containing a non-natural ratio of capsid proteins. Incorporation of a non-native sequence within the virus defines the viral vector as a “recombinant” vector, and hence a “rAAV vector.” A rAAV vector can include a heterologous polynucleotide (e.g., human codon-optimized gene encoding a human protein) encoding a desired protein or polypeptide. A recombinant vector sequence may be encapsidated or packaged into an AAV capsid and referred to as an “rAAV vector,” an “rAAV vector particle,” “rAAV viral particle” or simply a “rAAV.”

For the production of a rAAV vector, the desired ratio of VP1:VP2:VP3 is in the range of about 1:1:1 to about 1:1:100, such as in the range of about 1:1:2 to about 1:1:50, such as in the range of about 1:1:5 to about 1:1:20. Although the desired ratio of VP1:VP2 is 1:1, the ratio range of VP1:VP2 could vary from 1:50 to 50:1. The methods described herein provide for an analysis of the ratio of VP1:VP2:VP3, VP1:VP2, VP2:VP3, and/or VP1:VP3 as well as the characterization of any posttranslational modifications (PTMs) of those capsid components. The methods disclosed herein can distinguish and/or identify ratios of VP1:VP2, VP2:VP1, VP2:VP3, VP:3, VP3:VP1 and/or VP1:VP3 that are 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, 1:5, 1:5.1, 1:5.2, 1:5.3, 1:5.4, 1:5.5, 1:5.6, 1:5.7, 1:5.8, 1:5.9, 1:6, 1:6.1, 1:6.2, 1:6.3, 1:6.4, 1:6.5, 1:6.6, 1:6.7, 1:6.8, 1:6.9, 1:7, 1.71, 1:7.2, 1:7.3, 1:7.4, 1:7.5, 1:7.6, 1:7.7, 1:7.8, 1:7.9, 1:8, 1:8.1, 1:8.2, 1:8.3, 1:8.4, 1:8.5, 1:8.6, 1:8.7, 1:8.8, 1:8.9, 1:9 or higher (or any range derivable therein); these rations also apply to combinations thereof such as VP1:VP2:VP3 or VP3:VP2:VP1.

A viral capsid of an rAAV vector may be from a wild type AAV or a variant AAV such as AAV1, AAV2, AAV3, AAV3A, AAV3B, AAV4, AAV5, AAV6, AAV7, AAV8, AAV9, AAV10, AAVrh10, AAVrh74 (see WO2016/210170), AAV12, AAV2i8, AAV1.1, AAV2.5, AAV6.1, AAV6.3.1, AAV9.45, RHM4-1 (SEQ ID NO:5 of WO 2015/013313), RHM15-1, RHM15-2, RHM15-3/RHM15-5, RHM15-4, RHM15-6, AAV hu.26, AAV1.1, AAV2.5, AAV6.1, AAV6.3.1, AAV9,45, AAV2i8, AAV29G, AAV2,8G9, AVV-LK03, AAV2-TT, AAV2-TT-S312N, AAV3B-S312N, AAV avian AAV, bovine AAV, canine AAV, equine AAV, primate AAV, non-primate AAV, snake AAV, goat AAV, shrimp AAV, ovine AAV, and variants thereof (see, e.g., Fields, et al., Virology, volume 2, chapter 69 (4^{th} ed., Lippincott-Raven Publishers, which is hereby incorporated by reference). Capsids may be derived from a number of AAV serotypes disclosed in U.S. Pat. No. 7,906,111; Gao, et al. (2004). Journal of Virology, 78, 6381; Morris et al. (2004) Virology, 33, 375; WO 2013/063379; WO 2014/194132; and include true type AAV (AAV-TT) variants disclosed in WO 2015/121501, and RHM4-1, RHM15-1 through RHM15-6, and variants thereof, disclosed in WO 2015/013313, which are hereby incorporated by reference. Capsids may also be derived from AAV variants isolated from human CD34+ cell include AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14, and AAVHSC15 (Smith, et al. (2014). Molecular Therapy, 22(9), 1625-1634, which is hereby incorporated by reference). One skilled in the art would know there are likely other AAV variants not yet identified that perform the same or similar function. A full complement of AAV capsid proteins can include VP1, VP2, and VP3. The ORF comprising nucleotide sequences encoding AAV VP capsid proteins may comprise less than a full complement AAV capsid proteins or the full complement of AAV capsid proteins may be provided. All of the preceding rAAV viral capsids may be analyzed and characterized using the disclosed methods to determine the serotype and/or the heterogeneity.

In some embodiments, methods for the analysis and characterization by HILIC of ancestral AAV vectors for use in therapeutic in vivo gene therapy are described. Specifically, in silico-derived sequences may be synthesized de novo and characterized for biological activities. Prediction and synthesis of ancestral sequences, in addition to assembly into a rAAV vector, may be accomplished using methods described in WO 2015/054653, the contents of which are incorporated by reference herein. Notably, rAAV vectors assembled from ancestral viral sequences may exhibit reduced susceptibility to pre-existing immunity in human populations as compared to contemporary viruses or portions thereof. These vectors can be analyzed for serotype and heterogeneity by employing the present HILIC-MS methods.

In some embodiments, methods can be used to analyze and characterize a rAAV vector comprising a capsid protein encoded by a nucleotide sequence derived from more than one AAV serotype (e.g., wild type AAV serotypes, variant AAV serotypes)—which is referred to as a “chimeric vector” or “chimeric capsid” (See U.S. Pat. No. 6,491,907, the entire disclosure of which is incorporated herein by reference)—to determine the serotypes and heterogeneity of the rAAV vector. In some embodiments, a chimeric capsid protein is encoded by a nucleic acid sequence derived from 2, 3, 4, 5, 6, 7, 8, 9, 10 or more AAV serotypes. In some embodiments, a recombinant AAV vector includes a capsid sequence derived from e.g., AAV1, AAV2, AAV3, AAV3A, AAV3B, AAV4, AAV5, AAV6, AAV7, AAV8, AAV9, AAV10, AAV11, AAVrh74, AAVrh10, AAV2i8, or variant thereof, resulting in a chimeric capsid protein comprising a combination of amino acids from any of the foregoing AAV serotypes (see, Rabinowitz, et al. (2002). Journal of Virology, 76(2), 791-801, which is hereby incorporated by reference). Alternatively, a chimeric capsid can comprise a mixture of a VP1 from one serotype, a VP2 from a different serotype, a VP3 from yet a different serotype, and a combination thereof. For example, a chimeric virus capsid may include an AAV1 capsid protein or subunit and at least one AAV2 capsid protein or subunit. A chimeric capsid can, for example include an AAV capsid with one or more B19 virus capsid subunits, e.g., an AAV capsid protein or subunit can be replaced by a B19 virus capsid protein or subunit. For example, in one embodiment, the present method can be used to analyze and characterize an AAV capsid that has a VP3 subunit replaced with a VP2 subunit of B19 and thus determine the heterogeneity of the chimeric capsid as compared to a non-chimeric capsid.

In some embodiments, chimeric vectors have been engineered to exhibit altered tropism or tropism for a particular tissue or cell type. The term “tropism” refers to preferential entry of the virus into certain cell or tissue types and/or preferential interaction with the cell surface that facilitates entry into certain cell or tissue types. A “tropism profile” refers to a pattern of transduction of one or more target cells, tissues and/or organs. For example, an AAV capsid may have a tropism profile characterized by efficient transduction of muscle cells with only low transduction of, for example, brain cells. AAV tropism is generally determined by the specific interaction between distinct viral capsid proteins and their cognate cellular receptors (Lykken, et al. (2018). Journal of Neurodevelopmental Disorders, 10, 16). Once a virus or viral vector has entered a cell, sequences (e.g., heterologous sequences such as a transgene) carried by the vector genome (e.g., a rAAV vector genome) can be expressed. The methods described herein can be used to analyze and characterize the capsid components of these chimeric vectors in order to determine their serotype(s), heterogeneity, and/or tropism (predicted and/or based on factors known to influence tropism).

In some embodiments, methods described herein can be used to characterize rAAV vector preparations of various AAV serotypes and/or from chimeric capsids (e.g., AAV1, AAV2, AAV3 including AAV3A and AAV3B, AAV4, AAV5, AAV6, AAV7, AAV8, AAV9, AAV10, AAV12, AAVrh10, AAVrh74, avian AAV, bovine AAV, canine AAV, equine AAV, primate AAV, non-primate AAV, and ovine AAV, and recombinantly produced variants (e.g., capsid variants with insertions, deletions and substitutions, etc.), such as variants referred to as AAV2i8, NP4, NP22, NP66, AAVDJ, AAVDJ/8, AAVDJ/9, AAVLK03, RHM4-1, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14 and AAVHSC15). The methods described herein, can be used to monitor the manufacture of GMP clinical and commercial grade rAAV vectors to treat disease (e.g., DMD, Friedreich's Ataxia, Wilson Disease, etc.).

In some embodiments, AAV particle or rAAV particle containing complete and/or disrupted capsids, and/or denatured and/or non-denatured proteins can be loaded to the column, wherein the AAV or rAAV can be selected from AAV1, AAV2, AAV3, AAV3A, AAV3B, AAV4, AAV5, AAV6, AAV7, AAV8, AAV9, AAV10, AAVrh10, AAVrh74, AAV12, AAV2i8, NP4, NP22, NP66, AAVDJ, AAVDJ/8, AAVDJ/9, AAVLK03, AAV1.1, AAV2.5, AAV6.1, AAV6.3.1, AAV9.45, RHM4-1 (SEQ ID NO:5 of WO 2015/013313), RHM15-1, RHM15-2, RHM15-3/RHM15-5, RHM15-4, RHM15-6, AAV hu.26, AAV1.1, AAV2.5, AAV6.1, AAV6.3.1, AAV9,45, AAV2i8, AAV29G, AAV2,8G9, AVV-LK03, AAV2-TT, AAV2-TT-S312N, AAV3B-S312N, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14, AAVHSC15 and variants thereof. In some embodiments, one or more AAV particle described herein can be excluded.

The evaluation of the AAV particles may be part of a clinical or preclinical evaluation of the particles or it may be part of a quality control assessment of a production batch. The particles may be in a pharmaceutically acceptable or physiologically acceptable formulation.

II. HILIC Chromatography

First termed by Andrew J. Alpert, HILIC utilizes a polar stationary phase where a water layer is typically formed on-column. This is achieved by using a mobile phase that contains a relatively small amount of water in an organic solvent, typically acetonitrile (ACN). Polar analytes are retained by binding to the water layer and eluted with an increasing hydrophilic mobile phase such as water or methanol. Alpert, A. J. (1990). Journal of Chromatography A, 499, 177-196. Methods described herein can use HILIC chromatography to separate capsid proteins of a AAV capsid. AAV capsid can get denatured in the HILIC column and the component proteins can be eluted with the mobile phase in order of increasing polarity.

A. Sample Loading

In some embodiments, AAV particle can be loaded onto the HILIC column by direct injection of a load or load solution. The term “Load” or “load solution” can refer to a material (e.g., a viral capsid or a solution or suspension thereof) containing a product of interest (e.g., AAV particle, AAV capsid, rAAV capsid, or full rAAV vector) that is loaded onto a HILIC column. The load solution can contain a AAV particle. In some embodiments, a neat sample of the AAV particle can be loaded on the HILIC column. In some embodiments, the AAV capsid can be denatured prior to loading on to the HILIC column. In some embodiments, the AAV capsid is not denatured, or not reduced prior to loading onto the HILIC column. In some embodiments, the product of interest is a mixture of biological material, such as a protein mixture or a protein and nucleic acid mixture. In some embodiments, load solution can contain the product of interest in a solution, such as a buffer solution. In some embodiments, the load solution can contain the product of interest in phosphate-buffered saline (PBS) solution. In some embodiments, the load solution can contain the product of interest in an organic solvent, such as acetonitrile. In some embodiments, the load solution contains a denaturing agent, such as but not limited to an acid, a base, a reducing agent, an oxidizing agent, or a denaturing organic solvent. In some embodiments, the load solution does not contain a denaturing agent. In some embodiments, the load solution contains trifluoroacetic acid. In some embodiments, the load solution does not contain trifluoroacetic acid.

In some embodiments, the volume of the load or load solution loaded onto the column can be 0.01 vol. % to 100 vol. % or greater of the column. In some embodiments, the volume of the load is about, at least about, or at most about 0.01, 0.05, 0.1, 0.2, 0.3, 0.4, 0.5, 0.6, 0.7, 0.8, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 wt. % (or any range derivable therein) of the column. For example, in some embodiments, the volume of the load or load solution is 0.01, 0.05, 0.1, 0.2, 0.3, 0.4, 0.5, 0.6, 0.7, 0.8, 1, 1.5, 2, 2.5, 3, 3.5, 4, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μL for a column that has a volume of 100 μL. In some embodiments, the volume of the load is about, at least about, or at most about 0.01, 0.05, 0.1, 0.2, 0.3, 0.4, 0.5, 0.6, 0.7, 0.8, 1, 2, 3, 4, or 5, μL (or any range derivable therein).

The load concentration can be expressed as viral particles per milliliter (vp/mL), based on the viral particles present in the load or that were denatured to produce the load. In some embodiments, the load solution can have a load concentration of about, at least about, or at most about 1×10¹⁰, 2×10¹⁰, 3×10¹⁰, 3×10¹⁰, 4×10¹⁰, 5×1¹⁰, 6×10¹⁰, 7×10¹⁰, 8×10¹⁰, 9×10¹⁰, 1×10¹¹, 2×10¹¹, 3×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 3×10¹², 4×10¹², 5×10¹², 6×10¹², 7×10¹², 8×10¹², 9×10¹², 1×10¹³, 2×10¹³, 3×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 3×10¹⁴, 4×10¹⁴, or 5×10¹⁴ vp/mL (or any range derivable therein). In some embodiments, 0.1 μL to 5 μL, of load solution having a concentration of 5×10¹⁰ vp/mL to 1×10¹² vp/mL can be loaded onto a HILIC column having a volume of about 117 μL. In some embodiments, load solution having a volume higher than 5 μL, such as about, or at least about, 10, 20, 30, 40, 50, 60, 70, 80, 90, 100, 200, 300, 400, 500, 600, 700, 800, 900, 1000 or 10000 μL (or any range derivable therein), or higher than 10000 μL, can be loaded to the HILIC column. In some embodiments, load solution having a concentration higher than 5×10¹⁴ vp/mL, such as about, or at least about, 1×10¹⁵, 5×10¹⁵, 1×10¹⁶, 5×10¹⁶, 1×10¹⁷, 5×10¹⁷, 1×10¹⁵, 5×10¹⁵, 1×10¹⁹, 5×10¹⁹, or 1×10²⁰ vp/mL (or any range derivable therein), or higher than 1×10²⁰ vp/mL, can be loaded to the HILIC column.

B. Stationary Phase

The stationary phase in the HILIC column can be a resin or media suitable for separation of one AAV or rAAV capsid components from another (e.g., VP1, VP2, VP3 and PTMs thereof) using the methods described herein. A polar stationary phase can be used. Non-limiting examples of the stationary phase used can include bare silica, amide, aminopropyl, diol, zwitterionic (for example, sulfoalkylbetaine) phases, bonded phases upon silica, or bonded phases on organic polymer matrices. In some embodiments neutral stationary phases used in the methods described herein can contain polar functional groups that are in neutral form in the range of pH 3-8, a pH range usually used for the mobile phase in HILIC, and, thus, the retention is mainly supported by hydrophilic interactions. In some embodiments the HILIC stationary phases belong to this category, which comprises a large variety of functional groups. Stationary phases with functional groups such as the amide (TSK gel Amide-80), aspartamide (PolyHYDROXYETHYL A), diol (YMC-pack Diol), cross-linked diol (Luna HILIC), cyano (Alltima Cyano) and cyclodextrin (Nucleodex 11-0H) groups can be employed in the methods described herein. In some embodiments, a bridged ethylene hybrid amide (BEH Amide) can be used as the stationary phase for separating components of the AAV or rAAV capsid. In some embodiments, the stationary phase, such as BEH Amide containing stationary phase, can contain wide pores. In some embodiments, stationary phase with wide pores, such as having a pore size big enough to facilitate diffusion and reduce smearing, can be used. In some embodiments, a non-porous stationary phase, such as a non-porous stationary phase containing amide functional groups can be used. Particularly, a Waters ACQUITY UPLC BEH Column, available from Waters Corporation, 34 Maple Street, Milford, MA 01757, USA can be employed. The Waters ACQUITY Amide BEH column sizes that can be employed in the instant methods include 1.0×50 mm, 1.0×100 mm, 1.0×150 mm, 2.1×50 mm, 2.1×100 mm, 2.1×150 mm, 3.0×50 mm, 3.0×100 mm and 3.0×150 mm, optionally in conjunction with the use of a 2.1×5.0 mm precolumn.

C. Mobile Phase

The mobile phase used for elution and separation of AAV particles can be a mixture of water, an organic solvent, and a denaturing agent. In some embodiments, the mobile phase is a mixture of water, acetonitrile, and trifluoroacetic acid. The mobile phase used for elution and separation of AAV particles, can contain a 99.9 vol. % or more, or 99.95 vol. % or more, 99.99 vol. % or more or about 100 vol. % of a mixture of water, acetonitrile, and trifluoroacetic acid (TFA). AAV loaded HILIC column can be eluted with the mobile phase to obtain an eluent containing separated viral capsid protein.

In some embodiments, the starting water concentration in the mobile phase can be about, at least about, or at most about 25, 25.5, 26, 26.5, 27, 27.5, 28, 28.5, 29, 29.5 or 30 vol. % (or any range derivable therein). Starting water concentration refers to the water concentration in the mobile phase at the start of the elution, e.g. at elution run time 0. It was found starting water concentration effects capsid recovery and retention. A starting water concentration less than 25 vol. % can lead to high retention time and viral protein precipitation, and poor recovery. A starting water concentration greater than 30 vol. % can lead to relatively low retention time.

During elution the water concentration in the mobile phase can be increased. In some embodiments, the water concentration is increased at a rate about, at least about, or at most about 0.1, 0.2, 0.30, 0.32, 0.34, 0.36, 0.38, 0.4, 0.42, 0.44, 0.46, 0.48, 0.5, 0.52, 0.54, 0.56, 0.58, 0.60, 0.65, 0.70, 0.75, or 0.80 vol. %/min (or any range derivable therein). In some embodiments, the rate of increase can change over the elution time. For example, in some embodiments, the water concentration is increased at a rate of 0.3 vol. % per minute for the first half of the elusion, 0.6 vol. % per minutes for the next 40% of the elusion volume, and then 1 vol. % per minute for the last 10% of the elusion volume. It was found that difference in retention time of one or more component proteins can depend on the rate of increase of water concentration in the mobile phase.

TFA concentration in the mobile phase can be about, at least about, or at most about 10, 11, 12, 13, 14, 15, 16, 17, 18, 19, 20, 21, 22, 23, 24, 25, 26, 27, 28, 29, or 30 mM (or any range derivable therein). It was found that difference in retention time of one or more component proteins can depend on TFA concentration in the mobile phase. The TFA concentration can remain constant over the full volume of the elusion, or can change. In some embodiments, the TFA concentration remains constant at 20 mM over the full volume of the elusion. In some embodiments, TFA is added to the column as a solution of TFA in acetonitrile.

Acetonitrile can make up the remaining volume of the mobile phase. In some embodiments, another organic solvent is used instead of, or as a partial replacement for acetonitrile. Non-limiting examples of other organic solvents include aprotic solvents.

In some embodiments, starting water concentration in the mobile phase can be 25 to 30 volume percent, during elution water concentration of the mobile phase can be increased at a rate 0.3 to 0.6 volume percentage per minute, TFA concentration in the mobile phase is 10 mM to about 30 mM, and the remaining volume is acetonitrile. In some embodiments, starting water concentration in the mobile phase can be 27 to 29 volume percent, during elution water concentration of the mobile phase can be increased at a rate 0.35 to 0.45 vol. %/min., TFA concentration in the mobile phase is 17 mM to about 23 mM, and the remaining volume is acetonitrile. In some embodiments, starting water concentration in the mobile phase can be about 28 volume percent, during elution water concentration of the mobile phase can be increased at a rate about 0.4 vol. %/min., TFA concentration in the mobile phase is about 20 mM, and the remaining volume is acetonitrile.

The flow rate of the mobile phase during elution can be about, at least about, or at most about 0.010, 0.020, 0.030, 0.040, 0.050, 0.060, 0.070, 0.080, 0.090, 0.100, 0.105, 0.110, 0.115, 0.120, 0.125, 0.130, 0.135, 0.140, 0.145, 0.150, 0.155, 0.160, 0.170, 0.180, 0.190, 0.2, 0.25, 0.30, 0.35, 0.40, 0.45, 0.50, 0.55, 0.60, 0.65, 0.70, 0.75, 0.80, 0.85, 0.90, 0.95, or 1 mL/min (or any range derivable therein). In some embodiments, the flow rate of the mobile phase during elution can be about 0.140 mL/min. It was found the peak capacity, e.g., number of peaks in the HILIC chromatogram, can depend on the flow rate of the mobile phase. Relatively high peak capacity was obtained at flow rate 0.120 mL/min to 0.160 mL/min, or at about 0.14 mL/min. The flow rate can be the flow rate of the mobile phase through the column during elution.

D. Other HILIC Parameters

The run time for elution can be about, at least about, or at most about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 minutes (or any range derivable therein). The run time can refer to the total time the HILIC column is eluted using the mobile phase.

The column temperature during elution can be about, at least about, or at most about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60° C. (or any range derivable therein). It was found that difference in retention time of one or more component proteins can depend on the column temperature during elution.

III. Mass Spectrometry

Eluent obtained by elution of the HILIC column using the mobile phase can contain separated viral capsid proteins. Mass spectroscopy can be performed on at least a portion of the eluent to measure masses of one or more proteins, e.g. viral capsid proteins in the eluent. In some embodiments, the eluent can be directly flowed into the mass spectrometer used for mass spectrometry.

A “mass spectrometer” is an analytical instrument that can be used to determine the molecular weights of various substances, such as proteins and nucleic acids. It can also be used in some applications to determine the sequence of protein molecules and the chemical composition of virtually any material. Typically, a mass spectrometer comprises four parts: a sample inlet, an ionization source, a mass analyzer, and a detector. A sample is optionally introduced via various types of inlets, e.g., solid probe, GC, or LC, in gas, liquid, or solid phase. The sample is then typically ionized in the ionization source to form one or more ions. Ionization methods used can include but are not limited to electron ionization (E1), electrospray ionization (ESI), chemical ionization (CI), matrix-assisted laser desorption/ionization (MALDI). The resulting ions are introduced into and manipulated by the mass analyzer. Surviving ions are detected based on mass to charge ratio. In one embodiment, the mass spectrometer bombards the substance under investigation with an electron beam and quantitatively records the result as a spectrum of positive and negative ion fragments. Separation of the ion fragments is on the basis of mass to charge ratio of the ions. If all the ions are singly charged, this separation is essentially based on mass.

A. Electrospray Ionization (ESI)

ESI is a convenient ionization technique developed by Fenn and colleagues (Fenn, et al., (1989). Science, 246(4926), 64-71) that is used to produce gaseous ions from highly polar, mostly nonvolatile biomolecules, including lipids. The sample is injected as a liquid at low flow rates (1-10 μL/min) through a capillary tube to which a strong electric field is applied. The field generates additional charges to the liquid at the end of the capillary and produces a fine spray of highly charged droplets that are electrostatically attracted to the mass spectrometer inlet. The evaporation of the solvent from the surface of a droplet as it travels through the desolvation chamber increases its charge density substantially. When this increase exceeds the Rayleigh stability limit, ions are ejected and ready for MS analysis.

A typical conventional ESI source consists of a metal capillary of typically 0.1-0.3 mm in diameter, with a tip held approximately 0.5 to 5 cm (but more usually 1 to 3 cm) away from an electrically grounded circular interface having at its center the sampling orifice. (Kabarle, et al. (1993). Analytical Chemistry, 65(20), 972A-986A). A potential difference of between 1 to 5 kV (but more typically 2 to 3 kV) is applied to the capillary by power supply to generate a high electrostatic field (106 to 107 V/m) at the capillary tip. A sample liquid carrying the analyte to be analyzed by the mass spectrometer, is delivered to tip through an internal passage from a suitable source (such as from a chromatograph or directly from a sample solution via a liquid flow controller). By applying pressure to the sample in the capillary, the liquid leaves the capillary tip as small highly electrically charged droplets and further undergoes desolvation and breakdown to form single or multicharged gas phase ions in the form of an ion beam. The ions are then collected by the grounded (or negatively charged) interface plate and led through an orifice into an analyzer of the mass spectrometer. During this operation, the voltage applied to the capillary is held constant. Aspects of construction of ESI sources are described, for example, in U.S. Pat. Nos. 5,838,002; 5,788,166; 5,757,994; RE 35,413; 6,756,586, 5,572,023 and 5,986,258.

B. ESI/MS/MS

In ESI tandem mass spectroscopy (ESI/MS/MS), one is able to simultaneously analyze both precursor ions and product ions, thereby monitoring a single precursor product reaction and producing (through selective reaction monitoring (SRM)) a signal only when the desired precursor ion is present. When the internal standard is a stable isotope-labeled version of the analyte, this is known as quantification by the stable isotope dilution method. This approach has been used to accurately measure pharmaceuticals (Zweigenbaum, et al. (2000). Analytical Chemistry, 74, 2446) and bioactive peptides (Desiderio et al. (1996). Biopolymers, 40, 257). Newer methods are performed on widely available MALDI-TOF instruments, which can resolve a wider mass range and have been used to quantify metabolites, peptides, and proteins. Larger molecules such as peptides can be quantified using unlabeled homologous peptides as long as their chemistry is similar to the analyte peptide. Bucknall et al. (2002). J. Am. Soc. Mass Spectrometry, 13(9), 1015-27. Protein quantification has been achieved by quantifying tryptic peptides. Mirgorodskaya et al. (2000). Rapid Commun. Mass Spectrom., 14, 1226. Complex mixtures such as crude extracts can be analyzed, but in some embodiments sample cleanup is required. Gobom et al. (2000). Anal. Chem., 72, 3320. Desorption electrospray is a new associated technique for sample surface analysis.

C. SIMS

Secondary ion mass spectroscopy, or SIMS, is an analytical method that uses ionized particles emitted from a surface for mass spectroscopy at a sensitivity of detection of a few parts per billion. The sample surface is bombarded by primary energetic particles, such as electrons, ions (e.g., O, Cs), neutrals, or even photons, forcing atomic and molecular particles to be ejected from the surface, a process called sputtering. Since some of these sputtered particles carry a charge, a mass spectrometer can be used to measure their mass and charge. Continued sputtering permits measuring of the exposed elements as material is removed. This in turn permits one to construct elemental depth profiles. Although the majority of secondary ionized particles are electrons, it is the secondary ions which are detected and analysis by the mass spectrometer in this method.

D. LD-MS and LDLPMS

Laser desorption mass spectroscopy (LD-MS) involves the use of a pulsed laser, which induces desorption of sample material from a sample site-effectively, this means vaporization of sample off of the sample substrate. This method is usually only used in conjunction with a mass spectrometer, and can be performed simultaneously with ionization if one uses the right laser radiation wavelength.

When coupled with Time-of-Flight (TOF) measurement, LD-MS is referred to as LDLPMS (Laser Desorption Laser Photoionization Mass Spectroscopy). The LDLPMS method of analysis gives instantaneous volatilization of the sample, and this form of sample fragmentation permits rapid analysis without any wet extraction chemistry. The LDLPMS instrumentation provides a profile of the species present while the retention time is low and the sample size is small. In LDLPMS, an impactor strip is loaded into a vacuum chamber. The pulsed laser is fired upon a certain spot of the sample site, and species present are desorbed and ionized by the laser radiation. This ionization also causes the molecules to break up into smaller fragment-ions. The positive or negative ions made are then accelerated into the flight tube, being detected at the end by a microchannel plate detector. Signal intensity, or peak height, is measured as a function of travel time. The applied voltage and charge of the particular ion determines the kinetic energy, and the separation of fragments is due to different size causing different velocity. Each ion mass will thus have a different flight-time to the detector.

One can either form positive ions or negative ions for analysis. Positive ions are made from regular direct photoionization, but negative ion formation requires a higher powered laser and a secondary process to gain electrons. Most of the molecules that come off the sample site are neutrals, and thus can attract electrons based on their electron affinity. The negative ion formation process is less efficient than forming just positive ions. The sample constituents will also affect the outlook of negative ion spectra.

E. MALDI-TOF-MS

Since its inception and commercial availability, the versatility of MALDI-TOF-MS has been demonstrated convincingly by its extensive use for qualitative analysis. For example, MALDI-TOF-MS has been employed for the characterization of synthetic polymers, peptide and protein analysis (Zaluzec et al. (2000). Protein Expression and Purification, 6, 109, 1995; Roepstorff, et al. (2000). EXS, 88, 81), DNA and oligonucleotide sequencing, and the characterization of recombinant proteins. Recently, applications of MALDI-TOF-MS have been extended to include the direct analysis of biological tissues and single cell organisms with the aim of characterizing endogenous peptide and protein constituents. Li, et al. (2000). Trends in Biotechnology, 18, 151-160; Caprioli, et al. (1997). Analytical Chemistry, 69, 4751.

The properties that make MALDI-TOF-MS a popular qualitative tool—its ability to analyze molecules across an extensive mass range, high sensitivity, minimal sample preparation and rapid analysis times—also make it a potentially useful quantitative tool. MALDI-TOF-MS also enables non-volatile and thermally labile molecules to be analyzed with relative ease. It is therefore prudent to explore the potential of MALDI-TOF-MS for quantitative analysis in clinical settings, for toxicological screenings, as well as for environmental analysis. In addition, the application of MALDI-TOF-MS to the quantification of polypeptides (i.e., peptides and proteins) is particularly relevant.

F. Mass Analyzer

Mass analyzers separate the ions according to their mass-to-charge ratio. There are a variety of analyzers that can be used, including sector instruments, time-of-flight, quadrupole mass filter, three dimensional quadrupole ion trap, cylindrical ion trap, fourier transform ion cyclotron resonance etc.

Sector instruments—A sector field mass analyzer uses a static electric and/or magnetic field to affect the path and/or velocity of the charged particles in some way.

Time-of-flight—The time-of-flight (TOF) analyzer uses an electric field to accelerate the ions through the same potential, and then measures the time they take to reach the detector. If the particles all have the same charge, their kinetic energies will be identical, and their velocities will depend only on their masses. Ions with a lower mass will reach the detector first.

Quadrupole mass filter (QTOF)—Quadrupole mass analyzers use oscillating electrical fields to selectively stabilize or destabilize the paths of ions passing through a radio frequency (RF) quadrupole field created between 4 parallel rods. Only the ions in a certain range of mass/charge ratio are passed through the system at any time, but changes to the potentials on the rods allow a wide range of m/z values to be swept rapidly, either continuously or in a succession of discrete hops.

Ion traps—The quadrupole ion trap works on the same physical principles as the quadrupole mass analyzer, but the ions are trapped and sequentially ejected. The cylindrical ion trap mass spectrometer (CIT) is a derivative of the quadrupole ion trap where the electrodes are formed from flat rings rather than hyperbolic shaped electrodes.

In some embodiments, electrospray ionization (ESI) followed by tandem MS (MS/MS) can be used to measure masses of one or more viral capsid proteins. In some embodiments, matrix assisted laser desorption/ionization (MALDI) followed by time of flight (TOF) MS can be used to measure masses of one or more viral capsid proteins. In some embodiments, electrospray ionization (ESI) followed by quadrupole time of flight mass spectrometer (QTOF) MS can be used to measure masses of one or more viral capsid proteins. In some embodiments, masses of one or more viral capsid proteins were measured using ESI followed by QTOF, and the samples were analyzed in positive-ion mode. The detection range can be 600 to 5000 m/z.

IV. Capsid Proteins

All three-major species of viral capsid proteins (VP1, VP2 and VP3 and PTMs thereof) can be separated with high resolution using the HILIC methods described herein. In some embodiments, the most abundant capsid protein, VP3, is eluted away from capsid proteins VP1 and VP2. As a result of the high-resolution separation obtained with the present methods minor amounts of modified capsids can be separated, characterized, and/or identified. Modifications of the viral capsid components (i.e. modifications of VP1, VP2, or VP3) that can be detected using the present methods include but are not limited to truncation, additions, glycosylation, oxidation, phosphorylation, acetylation, deamidation, and disulfide bonding, and des-methionine, and des-threonine capsid proteins. Additional serotypes were screened under HILIC conditions that had been optimized for AAV9 and were found to have varying elution profiles with numerous modifications being identified. Thus, the presently claimed methods have been found to be advantageous for serotype determination and for determining the heterogeneity of AAV or rAAV particles.

Viral protein separated and characterized using the methods described herein can include one or more protein selected from VP1, VP2, VP3, and post translation modification (PTM)s thereof. PTMS of VP1, VP2, and/or VP3 can include independently glycosylation, disulfide bonding, acetylation, phosphorylation, deamidation, oxidation, des-methionine protein or des-threonine protein thereof, or any combination thereof. In some embodiments, PTMs of VP1 can include i) des-methionine and acetylated VP1, ii) des-methionine, acetylated and deamidated VP1, iii) des-methionine, acetylated, and oxidized VP1, iv) des-methionine, acetylated, and phosphorylated VP1, or any combinations thereof. In some embodiments, PTMs of VP2 can include, i) des-threonine VP2, ii) des-threonine and deamidated VP2, iii) des-threonine and oxidized VP2, iv) des-threonine and phosphorylated VP2, v) des-threonine and bisphosphorylated VP2, vi) desthreonine and acetylated VP2, vii) acetylated N-terminal methionine VP2, or any combinations thereof. In some embodiments, PTMs of VP3 can include i) des-methionine and acetylated VP3, ii) des-methionine, acetylated, and deamidated VP3, iii) des-methionine, acetylated, and oxidized VP3, iv) des-methionine, acetylated, and phosphorylated VP3. In some embodiments, masses of VP1, VP2, and/or VP3 can be measured. In some embodiments, masses of VP1, VP2, and VP3 can be measured. In some embodiments, masses of one or more proteins selected from i) VP1, i) des-methionine and acetylated VP1, iii) des-methionine, acetylated, and deamidated VP1, iv) des-methionine, acetylated, and oxidized VP1, and v) des-methionine, acetylated, and phosphorylated VP1, can be measured. In some embodiments, masses of one or more proteins selected from i) VP2, ii) des-threonine VP2, iii) des-threonine, and deamidated VP2, iv) des-threonine and oxidized VP2, v) des-threonine and phosphorylated VP2, vi) des-threonine and bisphosphorylated VP2, vii) des-threonine and acetylated VP2, and viii) acetylated N-terminal methionine VP2, can be measured. In some embodiments, masses of one or more protein selected from i) VP3, ii) des-methionine and acetylated VP3, iii) des-methionine, acetylated, and deamidated VP3, iv) des-methionine, acetylated, and oxidized VP3, v) des-methionine, acetylated, and phosphorylated VP3, can be measured. In some embodiments, one or more proteins described herein can be excluded.

In some embodiments, the masses of the one or more proteins can be compared with reference mass. The reference mass can be theoretical, predicted, and/or expected mass of a protein. In some embodiments, theoretical mass of a protein can be calculated or experimentally determined known mass of the protein. As discussed above, the masses of the one or more proteins can be indicative of the AAV serotype and/or heterogeneity. In some embodiments, amounts and/or relative amounts of the one or more proteins in the eluent can be determined. In some embodiments, the amount and/or relative amounts of the one or more proteins can be determined using mass spectroscopy and/or fluorescence spectroscopy of the eluent. In some embodiments, fluorescence can be determined at 275 nm excitation and 340 nm emission.

V. Definitions

Unless otherwise defined, all technical and scientific terms used herein have the meaning commonly understood by one of ordinary skill in the art to which this invention belongs. The terminology used herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise. The following terms have the meanings given:

As used herein, the terms “adeno-associated virus” and/or “AAV” refer to a parvovirus with a linear single-stranded DNA genome and variants thereof. The term covers all subtypes and both naturally occurring and recombinant forms, except where required otherwise.

The canonical AAV wild-type genome comprises 4681 bases (Berns, et al. (1987). Advances in Virus Research, 32, 243-307) and includes terminal repeat sequences (e.g., inverted terminal repeats (ITRs)) at each end which function in cis as origins of DNA replication and as packaging signals for the virus. The genome includes two large open reading frames, known as AAV replication (“AAV rep” or “rep”) and capsid (“AAV cap” or “cap”) genes, respectively. AAV rep and cap may also be referred to herein as AAV “packaging genes.” These genes code for the viral proteins involved in replication and packaging of the viral genome.

In wild type AAV, three capsid genes VP1, VP2, and VP3 overlap each other within a single open reading frame and alternative splicing leads to production of VP1, VP2 and VP3 capsid proteins (Grieger, et al. (2005). Journal of Virology, 79(15), 9933-9944). A single P40 promoter allows all three capsid proteins to be expressed at a ratio of about 1:1:10 for VP1, VP2, VP3, respectively, which complements AAV capsid production. More specifically, VP1 is the full-length protein, with VP2 and VP3 being increasingly shortened due to increasing truncation of the N-terminus. A well-known example is the capsid of AAV9 as described in U.S. Pat. No. 7,906,111, wherein VP1 comprises amino acid residues 1 to 736 of SEQ ID NO:123, VP2 comprises amino acid residues 138 to 736 of SEQ ID NO:123, and VP3 comprises amino acid residues 203 to 736 of SEQ ID NO:123. As used herein, the term “AAV Cap” or “cap” refers to AAV capsid proteins VP1, VP2 and/or VP3, and variants and analogs thereof. A second open reading frame of the capsid gene encodes an assembly factor, called assembly-activating protein (AAP), which is essential for the capsid assembly process (Sonntag, et al. (2011). Journal of Virology, 85(23), 12686-12697.

At least four viral proteins are synthesized from the AAV rep gene—Rep 78, Rep 68, Rep 52 and Rep 40—named according to their apparent molecular weights. As used herein, “AAV rep” or “rep” means AAV replication proteins Rep 78, Rep 68, Rep 52 and/or Rep 40, as well as variants and analogs thereof. As used herein, rep and cap refer to both wild type and recombinant (e.g., modified chimeric, and the like) rep and cap genes as well as the polypeptides they encode. In some embodiments, a nucleic acid encoding a rep will comprise nucleotides from more than one AAV serotype. For instance, a nucleic acid encoding a replication protein may comprise nucleotides from an AAV2 serotype and nucleotides from an AAV3 serotype (Rabinowitz, et al. (2002). Journal of Virology, 76(2), 791-801).

As used herein the terms “recombinant adeno-associated virus vector,” “rAAV” and/or “rAAV vector” refer to an AAV capsid comprising a vector genome. The vector genome comprises a polynucleotide sequence that is not, at least in part, derived from a naturally-occurring AAV (e.g., a heterologous polynucleotide not present in wild type AAV), and the rep and/or cap genes of the wild type AAV genome have been removed from the vector genome. Where the rep and/or cap genes of the AAV have been removed (and/or ITRs from an AAV have been added or remain), the nucleic acid within the AAV is referred to as the “vector genome.” Therefore, the term rAAV vector encompasses both a rAAV viral particle that comprises a capsid but does not comprise a complete AAV genome; instead the recombinant viral particle can comprise a heterologous, i.e., not originally present in the capsid, nucleic acid, hereinafter referred to as a vector genome. Thus, a “rAAV vector genome” (or “vector genome”) refers to a heterologous polynucleotide sequence (including at least one ITR) that may, but need not, be contained within an AAV capsid. A rAAV vector genome may be double-stranded (dsAAV), single-stranded (ssAAV) or self-complementary (scAAV). Typically, a vector genome comprises a heterologous (to the original AAV from which it is derived) nucleic acid often encoding a therapeutic transgene, a gene editing nucleic acid, and the like.

As used herein, the terms “rAAV vector,” “rAAV viral particle” and/or “rAAV vector particle” refer to an AAV capsid comprised of at least one AAV capsid protein (though typically all of the capsid proteins, e.g., VP1, VP2 and VP3, or variant thereof, of a AAV are present) and containing a vector genome comprising a heterologous nucleic acid sequence. These terms are to be distinguished from an “AAV viral particle” or “AAV virus” that is not recombinant wherein the capsid contains a virus genome encoding rep and cap genes and which AAV virus is capable of replicating if present in a cell also comprising a helper virus, such as an adenovirus and/or herpes simplex virus, and/or required helper genes therefrom. Thus, production of a rAAV vector particle necessarily includes production of a recombinant vector genome using recombinant DNA technologies, as such, which vector genome is contained within a capsid to form a rAAV vector, rAAV viral particle, or a rAAV vector particle.

rAAV vectors are referred to as “full,” a “full capsid,” “full vector” or a “fully packaged vector” when the capsid contains a complete vector genome, including a transgene. During production of rAAV vectors by host cells, vectors may be produced that have less packaged nucleic acid than the full capsids and contain, for example a partial or truncated vector genome. These vectors are referred to as “intermediates,” an “intermediate capsid,” a “partial” or a “partially packaged vector.” An intermediate capsid may also be a capsid with an intermediate sedimentation rate, that is a sedimentation rate between that of full capsids and empty capsids, when analyzed by analytical ultracentrifugation. Host cells may also produce viral capsids that do not contain any detectable nucleic acid material. These capsids are referred to as “empty(s),” or “empty capsids.”

As used herein, the term “associated with” refers to with one another, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example, by means of hydrogen bonds, van der Waals interaction, hydrophobic interactions, magnetism, and a combination thereof.

As used herein, the term “coding sequence” or “nucleic acid encoding” refers to a nucleic acid sequence which encodes a protein or polypeptide and denotes a sequence which is transcribed (in the case of DNA) and translated (in the case of mRNA) into a polypeptide in vitro or in vivo when placed under the control of (operably linked to) appropriate regulatory sequences. The boundaries of a coding sequence are generally determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences.

As used herein, the term “chimeric” refers to a viral capsid or particle, with capsid or particle sequences from different parvoviruses, such as different AAV serotypes, as described in Rabinowitz et al, U.S. Pat. No. 6,491,907, the disclosure of which is incorporated in its entirety herein by reference. See also Rabinowitz, et al. (2004). Journal of Virology, 78(9), 4421-4432. In some embodiments, a chimeric viral capsid is an AAV2.5 capsid which has the sequence of the AAV2 capsid with the following mutations: 263 Q to A; 265 insertion T; 705 N to A; 708 V to A; and 716 T to N. The nucleotide sequence encoding such capsid is defined as SEQ ID NO: 15 as described in WO 2006/066066. Chimeric AAV capsids can also include, but are not limited to, AAV2i8 described in WO 2010/093784, AAV2G9 and AAV8G9 described in WO 2014/144229, and AAV9.45 (Pulicherla, et al. (2011). Molecular Therapy, 19(6), 1070-1078), AAV-NP4, NP22 and NP66, AAV-LKO through AAV-LK019 described in WO 2013/029030, RHM4-1 and RHM15_1 through RHM5_6 described in WO 2015/013313, AAVDJ, AAVDJ/8, AAVDJ/9 described in WO 2007/120542.

As used herein, the term “eluate” refers to fluid exiting from a chromatography stationary phase (e.g., from the HILIC column media) (e.g., “eluting from the stationary phase”) comprised of mobile phase and material that passed through the stationary phase or was displaced from the stationary phase. In some embodiments, a stationary phase includes a resin or a media. The mobile phase may be a solution that has been loaded onto a column and is a gradient elution solution; a solution for regeneration of a stationary phase; a solution for sanitization of a stationary phase; a solution for washing; and combinations thereof.

As used herein, the term “flanked,” refers to a sequence that is flanked by other elements and indicates the presence of one or more flanking elements upstream and/or downstream, i.e., 5′ and/or 3′, relative to the sequence. The term “flanked” is not intended to indicate that the sequences are necessarily contiguous. For example, there may be intervening sequences between a nucleic acid encoding a transgene and a flanking element. A sequence (e.g., a transgene) that is “flanked” by two other elements (e.g., ITRs), indicates that one element is located 5′ to the sequence and the other is located 3′ to the sequence; however, there may be intervening sequences there between.

As used herein, the term “fragment” refers to a material or entity that has a structure that includes a discrete portion of the whole but lacks one or more moieties found in the whole. In some embodiments, a fragment consists of a discrete portion. In some embodiments, a fragment consists of or comprises a characteristic structural element or moiety found in the whole. In some embodiments, a polymer fragment comprises, or consists of, at least or at most 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., amino acid residues, nucleotides) found in the whole polymer (or any range derivable therein). Representative fragments that can be analyzed and characterized using the HILIC-MS methods described herein to determine their serotype and/or heterogeneity include fragments of the VP1, VP2 and VP3 capsid components of AAV or rAAV. For example, the instantly claimed methods have been found to be useful for determining heterogeneity of the capsid components in which certain components are des-methionine VP1 or des-threonine VP2.

As used herein, the term “null capsid” refers to a capsid produced intentionally to lack a vector genome. Such null a capsid may be produced by transfection of a host cell with a rep/cap and a helper plasmid, but not a plasmid that comprises the transgene cassette sequence, also known as a vector plasmid.

As used herein, the term “gene” refers to a polynucleotide containing at least one open reading frame that is capable of encoding a particular polypeptide or protein after being transcribed and translated. “Gene transfer” or “gene delivery” refers to methods or systems for reliably inserting foreign DNA into host cells. Such methods can result in transient expression of non-integrated transferred DNA, extrachromosomal replication and expression of transferred replicons (e.g. episomes), and/or integration of transferred genetic material into the genomic DNA of host cells.

As used herein, the term “gradient elution” refers to application of a mixture of at least two different solvents or solutions to a chromatography stationary phase (an appropriate HILIC stationary phase). Over the course of the gradient elution, a percentage of one mobile phase component (solvent or solution) is varied in a manner inversely proportional to a percentage of a second mobile phase component (solvent or solution). For example, at the start of a gradient elution, the percentage of mobile phase A (e.g., water) is about 28% and the percentage of gradient mobile phase B (e.g., acetonitrile) is about 62% with a mobile phase C (5% TFA in water) being eluted at a rate such that the overall eluent has a TFA concentration of 20 mM. The percentage of mobile phase A (water) is then gradually increased and the percentage of mobile phase B (acetonitrile) is inversely decreased while the mobile phase C (5% TFA) is held constant such that the overall concentration in the eluent is 20 mM. In some embodiments, AAV capsids or rAAV capsids (e.g., full, intermediate, empty) are bound to a stationary phase during a step of loading a sample (neat, solution or suspension) comprising the AAV or rAAV capsid onto an stationary HILIC phase. During a gradient elution, as the percentage of mobile phase A (water) increases, the AAV or rAAV capsid components elute from the column and can then be further characterized by mass spectrometry. In some embodiments, VP3 can elute from the HILIC column first followed by VP1 and then VP2. Elution of the AAV or rAAV components can be monitored using on-line UV trace, off-line UV methods, etc., and wherein the absorbance is measured at one or more wavelengths (e.g., 260 nm and/or 280 nm).

As used herein, the term “heterologous” refers to a nucleic acid inserted into a vector (e.g., rAAV vector) for purposes of vector mediated transfer/delivery of the nucleic acid into a cell. Heterologous nucleic acids are typically distinct from the vector (e.g., AAV) nucleic acid, that is, the heterologous nucleic acid is non-native with respect to the viral (e.g., AAV) nucleic acid. Once transferred or delivered into a cell, a heterologous nucleic acid, contained within a vector, can be expressed (e.g., transcribed and translated if appropriate). Alternatively, a transferred or delivered heterologous nucleic acid in a cell, contained within the vector, need not be expressed. Although the term “heterologous” is not always used herein in reference to a nucleic acid, reference to a nucleic acid even in the absence of the modifier “heterologous” is intended to include a heterologous nucleic acid.

As used herein, the term “homologous,” or “homology,” refers to two or more reference entities (e.g., nucleotide or polypeptide sequences) that share at least partial identity over a given region or portion. For example, when an amino acid position in two peptides is occupied by identical amino acids, the peptides are homologous at that position. Notably, a homologous peptide will retain activity or function associated with the unmodified or reference peptide and the modified peptide will generally have an amino acid sequence “substantially homologous” with the amino acid sequence of the unmodified sequence. When referring to a polypeptide, nucleic acid or fragment thereof, “substantial homology” or “substantial similarity,” means that when optimally aligned with appropriate insertions or deletions with another polypeptide, nucleic acid (or its complementary strand) or fragment thereof, there is sequence identity in at least about 95% to 99% of the sequence. The extent of homology (identity) between two sequences can be ascertained using computer program or mathematical algorithm. Such algorithms that calculate percent sequence homology (or identity) generally account for sequence gaps and mismatches over the comparison region or area. Exemplary programs and algorithms are provided below.

As used herein, the terms “host cell,” “host cell line,” and “host cell culture” are used interchangeably and refers to a cell into which an exogenous nucleic acid has been introduced, and includes the progeny of such a cell. A host cell includes a “transfectant,” “transformant,” “transformed cell,” and “transduced cell,” which includes the primary transfected, transformed or transduced cell, and progeny derived therefrom, without regard to the number of passages. In some embodiments, a host cell is a packaging cell for production of a rAAV vector.

As used herein, the terms “inverted terminal repeat,” “ITR,” “terminal repeat,” and “TR” refer to palindromic terminal repeat sequences at or near the ends of the AAV virus genome, comprising mostly complementary, symmetrically arranged sequences. These ITRs can fold over to form T-shaped hairpin structures that function as primers during initiation of DNA replication. They are also needed for viral genome integration into host genome, for the rescue from the host genome; and for the encapsidation of viral nucleic acid into mature virions. The ITRs are required in cis for vector genome replication and its packaging into viral particles. “5′ ITR” refer to the ITR at the 5′ end of the AAV genome and/or 5′ to a recombinant transgene. “3′ ITR” refers to the ITR at the 3′ end of the AAV genome and/or 3′ to a recombinant transgene. Wild-type ITRs are approximately 145 bp in length. A modified, or recombinant ITR, may comprise a fragment or portion of a wild-type AAV ITR sequence. One of ordinary skill in the art will appreciate that during successive rounds of DNA replication ITR sequences may swap such that the 5′ ITR becomes the 3′ ITR, and vice versa. In some embodiments, at least one ITR is present at the 5′ and/or 3′ end of a recombinant vector genome such that the vector genome can be packaged into a capsid to produce a rAAV vector (also referred to herein as “rAAV vector particle” or “rAAV viral particle”) comprising the vector genome.

The ITRs are required in cis for vector genome replication and its packaging into viral particles. “5′ ITR” refer to the ITR at the 5′ end of the AAV genome and/or 5′ to a recombinant transgene. “3′ ITR” refers to the ITR at the 3′ end of the AAV genome and/or 3′ to a recombinant transgene. Wild-type ITRs are approximately 145 bp in length. A modified, or recombinant ITR, may comprise a fragment or portion of a wild-type AAV ITR sequence. One of ordinary skill in the art will appreciate that during successive rounds of DNA replication ITR sequences may swap such that the 5′ ITR becomes the 3′ ITR, and vice versa.

As used herein, the terms “nucleic acid sequence,” “nucleotide sequence,” and “polynucleotide” refer interchangeably to any molecule composed of or comprising monomeric nucleotides connected by phosphodiester linkages. A nucleic acid may be an oligonucleotide or a polynucleotide. Nucleic acid sequences are presented herein in the direction from the 5′ to the 3′ direction.

As used here, the term “nucleic acid construct,” refers to a non-naturally occurring nucleic acid molecule resulting from the use of recombinant DNA technology (e.g., a recombinant nucleic acid). A nucleic acid construct is a nucleic acid molecule, either single or double stranded, which has been modified to contain segments of nucleic acid sequences, which are combined and arranged in a manner not found in nature. A nucleic acid construct may be a “vector” (e.g., a plasmid, a rAAV vector genome, an expression vector, etc.), that is, a nucleic acid molecule designed to deliver exogenously created DNA into a host cell.

As used herein, the term “operably linked” refers to a linkage of nucleic acid sequence (or polypeptide) elements in a functional relationship. A nucleic acid is operably linked when it is placed into a functional relationship with another nucleic acid sequence. For instance, a promoter or other transcription regulatory sequence (e.g., an enhancer) is operably linked to a coding sequence if it affects the transcription of the coding sequence. In some embodiments, operably linked means that nucleic acid sequences being linked are contiguous. In some embodiments, operably linked does not mean that nucleic acid sequences are contiguously linked, rather intervening sequences are between those nucleic acid sequences that are linked.

As used herein, the term “pharmaceutically acceptable” and “physiologically acceptable” refers to a biologically acceptable formulation, gaseous, liquid or solid, or mixture thereof, which is suitable for one or more routes of administration, in vivo delivery or contact.

As used herein, the terms “polypeptide,” “protein,” “peptide” or “encoded by a nucleic acid sequence” (i.e., encode by a polynucleotide sequence, encoded by a nucleotide sequence) refer to full-length native sequences, as with naturally occurring proteins, as well as functional subsequences, modified forms or sequence variants so long as the subsequence, modified form or variant retains some degree of functionality of the native full-length protein. In methods and uses of the disclosure, such polypeptides, proteins and peptides encoded by the nucleic acid sequences can be but are not required to be identical to the endogenous protein that is defective, or whose expression is insufficient, or deficient in a subject treated with gene therapy.

As used herein, the term “recombinant,” refers to a vector, polynucleotide (e.g., a recombinant nucleic acid), polypeptide or cell that is the product of various combinations of cloning, restriction or ligation steps (e.g., relating to a polynucleotide or polypeptide comprised therein), and/or other procedure that results in a construct that is distinct from a product found in nature. A recombinant virus or vector (e.g., rAAV vector) comprises a vector genome comprising a recombinant nucleic acid (e.g., a nucleic acid comprising a transgene and one or more regulatory elements). The terms respectively include replicates of the original polynucleotide construct and progeny of the original virus construct.

As used herein, the term “substantially” refers to the qualitative condition of exhibition of total or near-total extent or degree of a characteristic or property of interest. One of ordinary skill in the art will understand that biological and chemical phenomena rarely, if ever, go to completion and/or proceed to completeness or achieve or an absolute result. The term “substantially” is therefore used herein to capture the potential lack of completeness inherent in many biological and chemical phenomena.

As used herein, the term “therapeutic polypeptide” is a peptide, polypeptide or protein (e.g., enzyme, structural protein, transmembrane protein, transport protein) that may alleviate or reduce symptoms that result from an absence or defect in a protein in a target cell (e.g., an isolated cell) or organism (e.g., a subject). A therapeutic polypeptide or protein encoded by a transgene is one that confers a benefit to a subject, e.g., to correct a genetic defect, to correct a deficiency in a gene related to expression or function. Similarly, a “therapeutic transgene” is the transgene that encodes the therapeutic polypeptide. In some embodiments, a therapeutic polypeptide, expressed in a host cell, is an enzyme expressed from a transgene (i.e., an exogenous nucleic acid that has been introduced into the host cell).

As used herein, the term “transgene” is used to mean any heterologous polynucleotide for delivery to and/or expression in a host cell, target cell or organism (e.g., a subject). Such “transgene” may be delivered to a host cell, target cell or organism using a vector (e.g., rAAV vector). A transgene may be operably linked to a control sequence, such as a promoter. It will be appreciated by those of skill in the art that expression control sequences can be selected based on an ability to promote expression of the transgene in a host cell, target cell or organism. Generally, a transgene may be operably linked to an endogenous promoter associated with the transgene in nature, but more typically, the transgene is operably linked to a promoter with which the transgene is not associated in nature. Such a non-endogenous promoter can include a CBh promoter or a muscle specific promoter, among many others known in the art.

A nucleic acid of interest can be introduced into a host cell by a wide variety of techniques that are well-known in the art, including transfection and transduction.

“Transfection” is generally known as a technique for introducing an exogenous nucleic acid into a cell without the use of a viral vector. As used herein, the term “transfection” refers to transfer of a recombinant nucleic acid (e.g., an expression plasmid) into a cell (e.g., a host cell) without use of a viral vector. A cell into which a recombinant nucleic acid has been introduced is referred to as a “transfected cell.” A transfected cell may be a host cell (e.g., a CHO cell, Pro10 cell, HEK293 cell) comprising an expression plasmid/vector for producing a recombinant AAV vector. In some embodiments, a transfected cell (e.g., a packing cell) may comprise a plasmid comprising a transgene, a plasmid comprising an AAV rep gene, and an AAV cap gene, and a plasmid comprising a helper gene. Many transfection techniques are known in the art, which include, but are not limited to, electroporation, calcium phosphate precipitation, microinjection, cationic or anionic liposomes, and liposomes in combination with a nuclear localization signal.

As used herein, the term “transduction” refers to transfer of a nucleic acid (e.g., a vector genome) by a viral vector (e.g., rAAV vector which can be analyzed by the methods described herein) to a cell (e.g., a target cell, e.g., a muscle cell). In some embodiments, a gene therapy includes transducing a vector genome comprising a modified nucleic acid encoding a human gene, or a fragment thereof, into a cell, such as a muscle cell. A cell into which a transgene has been introduced by a virus or a viral vector is referred to as a “transduced cell.” In some embodiments, a transduced cell is an isolated cell and transduction occurs ex vivo. In some embodiments, a transduced cell is a cell within an organism (e.g., a subject) and transduction occurs in vivo. A transduced cell may be a target cell of an organism which has been transduced by a recombinant AAV vector such that the target cell of the organism expresses a polynucleotide (e.g., a transgene, e.g., a modified nucleic acid encoding a human protein, or a fragment thereof).

Cells that may be transduced include a cell of any tissue or organ type, or any origin (e.g., mesoderm, ectoderm or endoderm). Non-limiting examples of cells include liver (e.g., hepatocytes, sinusoidal endothelial cells), pancreas (e.g., beta islet cells, exocrine), lung, central or peripheral nervous system, such as brain (e.g., neural or ependymal cells, oligodendrocytes) or spine, kidney, eye (e.g., retinal), spleen, skin, thymus, testes, lung, diaphragm, heart (cardiac), muscle or psoas, or gut (e.g., endocrine), adipose tissue (white, brown or beige), muscle (e.g., fibroblasts, myocytes), synoviocytes, chondrocytes, osteoclasts, epithelial cells, endothelial cells, salivary gland cells, inner ear nervous cells or hematopoietic (e.g., blood or lymph) cells. Additional examples include stem cells, such as pluripotent or multipotent progenitor cells that develop or differentiate into liver (e.g., hepatocytes, sinusoidal endothelial cells), pancreas (e.g., beta islet cells, exocrine cells), lung, central or peripheral nervous system, such as brain (e.g., neural or ependymal cells, oligodendrocytes) or spine, kidney, eye (e.g., retinal), spleen, skin, thymus, testes, lung, diaphragm, heart (cardiac), muscle or psoas, or gut (e.g., endocrine), adipose tissue (white, brown or beige), muscle (e.g., fibroblast, myocytes), synoviocytes, chondrocytes, osteoclasts, epithelial cells, endothelial cells, salivary gland cells, inner ear nervous cells or hematopoietic (e.g., blood or lymph) cells.

As used herein, the term “vector” refers to a plasmid, virus (e.g., a rAAV), cosmid, or other vehicle that can be manipulated by insertion or incorporation of a nucleic acid (e.g., a recombinant nucleic acid). A vector can be used for various purposes including, e.g., genetic manipulation (e.g., cloning vector), to introduce/transfer a nucleic acid into a cell, to transcribe or translate an inserted nucleic acid in a cell. In some embodiments a vector nucleic acid sequence contains at least an origin of replication for propagation in a cell. In some embodiments, a vector nucleic acid includes a heterologous nucleic acid sequence, an expression control element(s) (e.g., promoter, enhancer), a selectable marker (e.g., antibiotic resistance), a poly-adenosine (polyA) sequence and/or an ITR. In some embodiments, when delivered to a host cell, the nucleic acid sequence is propagated. In some embodiments, when delivered to a host cell, either in vitro or in vivo, the cell expresses the polypeptide encoded by the heterologous nucleic acid sequence. In some embodiments, when delivered to a host cell, the nucleic acid sequence, or a portion of the nucleic acid sequence is packaged into a capsid. A host cell may be an isolated cell or a cell within a host organism. In addition to a nucleic acid sequence (e.g., transgene) which encodes a polypeptide or protein, additional sequences (e.g., regulatory sequences) may be present within the same vector (i.e., in cis to the gene) and flank the gene. In some embodiments, regulatory sequences may be present on a separate (e.g., a second) vector which acts in trans to regulate the expression of the gene. Plasmid vectors may be referred to herein as “expression vectors.”

As used herein, the term “vector genome” refers to a nucleic acid that is packaged/encapsidated in an AAV capsid to form a rAAV vector. Typically, a vector genome includes a heterologous polynucleotide sequence (e.g., a transgene, regulatory elements, etc.) and at least one ITR. In cases where a recombinant plasmid is used to construct or manufacture a recombinant vector (e.g., rAAV vector), the vector genome does not include the entire plasmid but rather only the sequence intended for delivery by the viral vector. This non-vector genome portion of the recombinant plasmid is referred to as the “plasmid backbone,” which is important for cloning. selection and amplification of the plasmid, a process that is needed for propagation of recombinant viral vector production, but which is not itself packaged or encapsidated into a rAAV vector. Typically, the heterologous sequence to be packaged into the capsid is flanked by the ITRs such that when cleaved from the plasmid backbone, it is packaged into the capsid.

As used herein, the term “viral vector” generally refers to a viral particle that functions as a nucleic acid delivery vehicle and which comprises a vector genome (e.g., comprising a transgene which has replaced the wild type rep and cap) packaged within the viral particle (i.e., capsid) and includes, for example, lenti- and parvo-viruses, including AAV serotypes and variants (e.g., rAAV vectors). As noted elsewhere herein, a recombinant viral vector does not comprise a virus genome with a rep and/or a cap gene; rather, these sequences have been removed to provide capacity for the vector genome to carry a transgene of interest.

The following examples describe the methods of the present invention and are to be construed in a non-limiting manner.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1

### Sample Preparation, General HILIC Conditions, Mass Spectrometry Conditions

As described in this example and those following, HILIC conditions were evaluated and optimized for AAV9. Additionally, the elution profiles for AAV1, AAV2, AAV5, AAV6, and AAV8 capsid proteins were obtained.

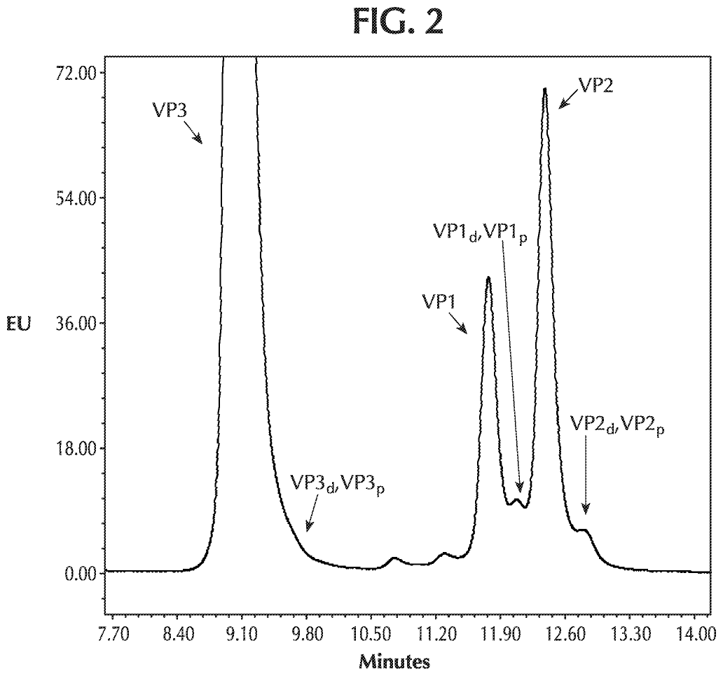

A method for the separation and identification AAV capsids and their associated post translational modifications (PTMs) has been developed. The effects of mobile phase starting conditions, elution gradient, flow rate, column temperature, and column load on AAV9 capsid separations by HILIC were evaluated. Under optimized conditions (see Table 5), VP3 elutes first, followed by VP1 and then VP2 (see FIG. 1). Minor species have been identified to be PTMs, consisting of a mixture of deamidation and phosphorylation, and truncations (see FIG. 2).

It was found that H₂O content of the starting mobile phase can influence the separation of capsid proteins in some embodiments. Other factors that can be used to improve resolution include TFA concentration, elution gradient, flow rate, and column temperature.

### A. Reagents and Materials

Acetonitrile (Optima LC/MS Grade, Fisher Chemical) and Trifluoroacetic Acid (Pierce LC/MS Grade, Thermo Scientific) were purchased from Fisher Scientific, Hampton NH. MilliQH₂O was purified from a Milli-Q IQ 7000 water purification system purchased from Millipore Sigma, Burlington MA. Multiple AAV serotypes were used for this research. For initial HILIC screening and development, AAV9 containing a therapeutic transgene was used (9.15×10¹³ vp/mL). Chromatographic profiles were also obtained for additional AAV serotypes containing no transgene. Empty AAV1, AAV2, AAV5, AAV6, AAV8, AAV9 capsids were purchased from Virovek Inc (Hayward, CA) at a concentration of 1×10¹⁴ vg/mL.

### B. Viral Sample Preparation

AAV capsids were not reduced or otherwise treated prior to analysis. AAV9 samples were injected neat unless otherwise indicated. Additional empty capsids (as described above: AAV1, AAV2, AAV5, AAV6, AAV8, AAV9) were diluted to 2.5E13 vg/mL in PBS and injected immediately.

### C. HILIC Chromatographic Conditions

HILIC optimization was carried out using the following general method. An amide HILIC column (Waters Acquity UPLC Glycoprotein BEH Amide, 300 Å, 1.7 μm) in a custom column format (1 mm×150 mm) was used to allow direct flow into a mass spectrometer. Different mobile phase compositions were screened by mixing stock solutions of acetonitrile (ACN), H₂O, and 5 wt. % Trifluoroacetic Acid (TFA) in water on an Acquity H-Class Ultra High Performance Liquid Chromatography (UHPLC) system (Waters Corporation, Millford, MA). Flow rates (0.060-0.160 mL/min), elution gradient (0.400-0.600% H₂O/min), and column temperatures (25-40° C.) were evaluated as described below. Unless noted, non-reduced AAV9 was injected at a volume of 0.5 uL. Capsid elution was monitored by fluorescence detection (275 nm excitation, 340 nm emission). After each injection, the column was cleaned for 10 column volumes (90 wt. H₂O, 10 wt. % ACN), and regenerated at starting conditions for 25 column volumes.

### D. Mass Spectrometry Conditions

Capsid proteins (protomers) from AAV serotypes were denatured on column and chromatographically separated on a Waters H-Class quaternary Ultra High Performance Liquid Chromatography (UHPLC) system using the previously referenced HILIC gradient and analytical column.

The Waters H-Class UHPLC was coupled to a Bruker maXis II mass spectrometer. Samples were analyzed in positive-ion mode with a detection range of 600-5000 m/z. The instrument was calibrated by infusing Agilent Tune Mix for ESI-QTOF MS instruments. Table 1 below describes the MS conditions used.

TABLE 1
Mass Spectrometry Conditions

| Parameter | Conditions | |
|---|---|---|
| Nebulizer Pressure | 1.6 | Bar |
| Dry Gas | 8.0 | l/min |
| Dry Gas Temperature | 200° | C. |
| Capillary Voltage | 4,500 | V |
| Funnel RF | 400 | Vpp |
| isCID | 45 | eV |
| Multipole RF | 400 | Vpp |
| Quadrupole Ion Energy | 6 | eV |
| Collision Energy | 15 | eV |
| Collision RF | 800 | Vpp |
| Transfer Time | 130 | μs |

### E. Data Processing and Analysis

Chromatographic data were processed by Empower 3 Chromatography Software (Waters Corporation). Data were visualized using Minitab (State College, PA).

Mass spectrometry data were processed with Bruker DataAnalysis software (4.4, Bruker Daltonics, Bremen, Germany) and individual observed capsid proteins masses were compared to theoretical capsid protein masses. Monoisotopic masses were determined for species with a molecular weight under 50,000 Da, while average masses were reported on species over 50,000 Da. The theoretical average masses were calculated with PAWS software (2000.06.08, Genomic Solutions, Ann Arbor, MI) for FIX AAV capsid proteins and proteolytic fragments thereof. The theoretical monoisotopic masses were calculated with Sequence Editor (3.2, Bruker Daltonics, Bremen, Germany).

### Example 2

### Evaluation of Mobile Phase Starting Conditions

Using the HILIC general method described above, a full factorial analysis was performed to evaluate TFA concentrations ranging from 10 mM to 50 mM in the mobile phase and an initial water content ranging from 10 wt. % to 35 wt. % in the mobile phase (Table 2). A total of 30 separate conditions were evaluated (5×6 matrix, 5 different TFA concentrations (10, 20, 30, 40 and 50 mM) in the mobile phase were evaluated at each starting water percentage (10, 15, 20, 25, 30 and 35 wt. % water) in the mobile phase. The remainder of the content of the mobile phase was ACN.

TABLE 2
Water Content at Starting Conditions and Final
Conditions at Different TFA Concentrations

| TFA | Starting H₂O (wt. %) in the mobile phase | | | | | |
|---|---|---|---|---|---|---|
| (mM) | 10 | 15 | 20 | 25 | 30 | 35 |
| 10 | × | × | × | × | × | × |
| 20 | × | × | × | × | × | × |
| 30 | × | × | × | × | × | × |
| 40 | × | × | × | × | × | × |
| 50 | × | × | × | × | × | × |
| | Final H₂O (wt. %) in the mobile phase | | | | | |
| | 40 | 45 | 50 | 55 | 60 | 65 |

At each condition (each combination of TFA concentration and initial weight percentage H₂O), the elution gradient was increased at 0.5 wt. % H₂O/min for 60 minutes at a flow rate of 0.100 mL/min and at column temperature 30° C. This resulted in final water weight percentages of 40, 45, 50, 55, 60 and 65 in the mobile phase based on the initial water weight percentages of 10, 15, 20, 25, 30, and 35 in the mobile phase, respectively.

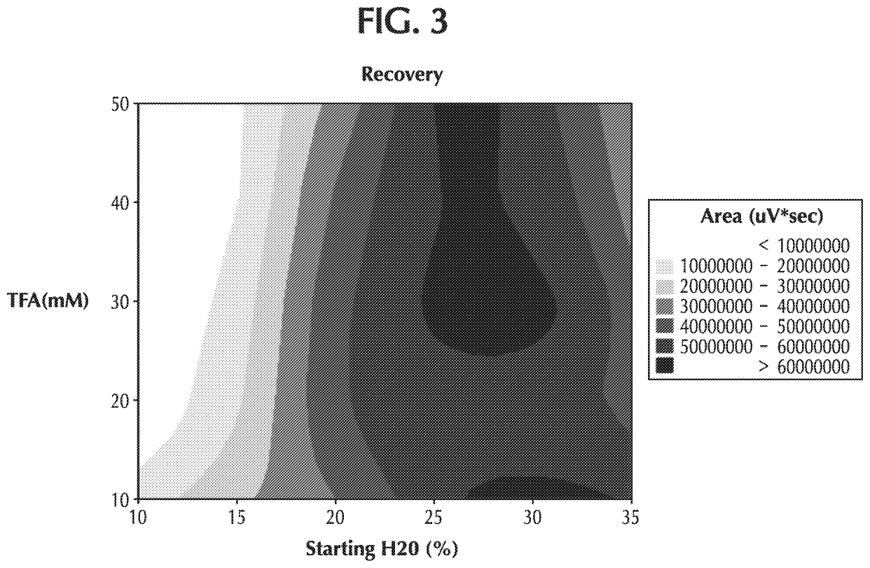

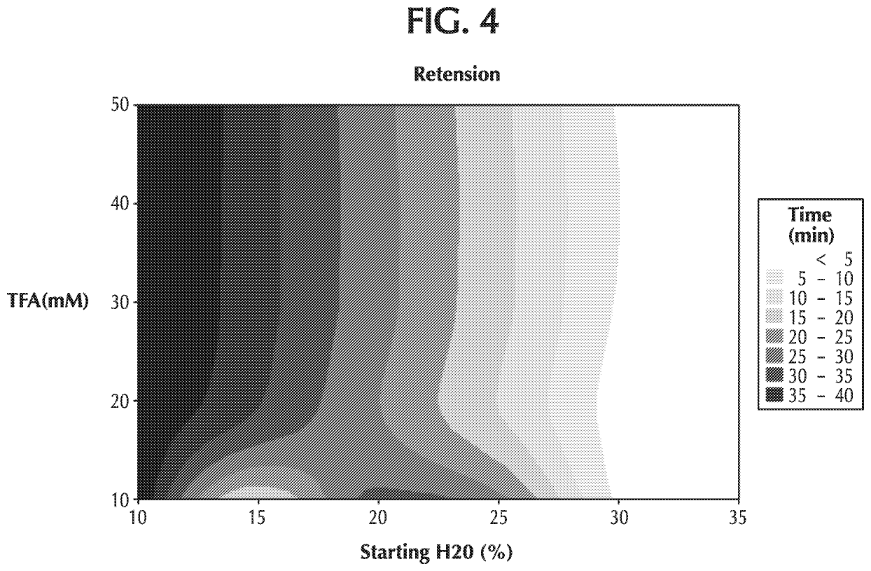

It was found that the Initial water content has a large and differing effect on capsid recovery and retention. Capsid recovery was highest when injecting at 28 wt. % H₂O in the mobile phase (see FIG. 3), for a final H₂O concentration of 58 wt. % in the mobile phase. Decreasing water content at loading resulted in a loss of capsid retained on the column. As expected, increasing the initial water content has the opposite effect on retention (see FIG. 4). Capsids were retained for greater than 40 minutes when starting at 10 wt. % H₂O and had a retention time less than 5 minutes when starting at 30 wt. % H₂O. Balance between capsid recovery and retention time can be made for an effective separation.

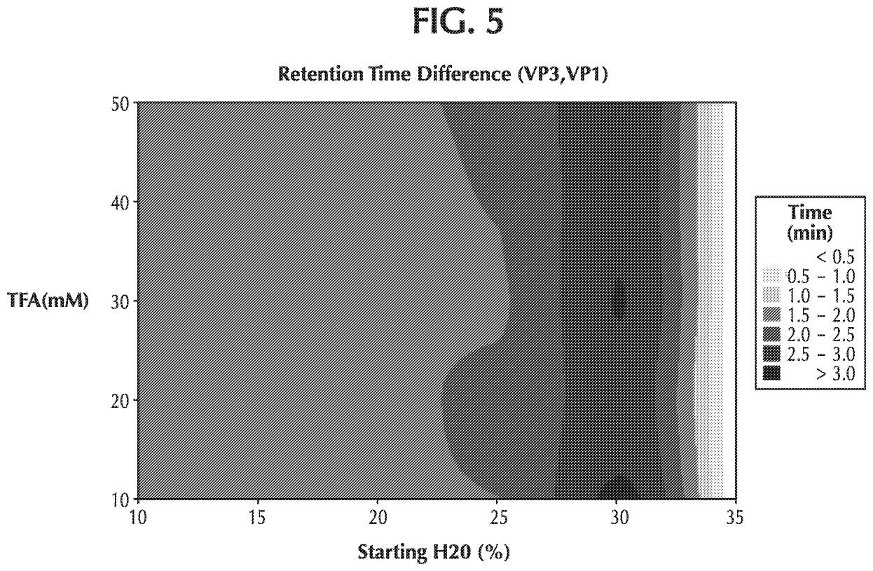

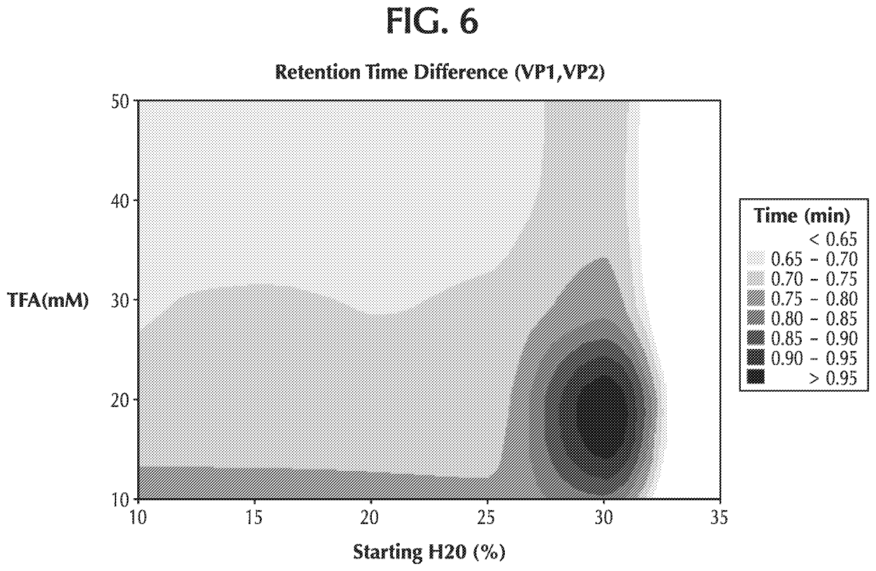

It was also found that TFA concentration has a lesser effect on capsid recovery and retention time but modulates the separation between VP1, VP2, and VP3. At a starting water content between 28 to 32 wt. % H₂O in the mobile phase, VP3 and VP1 had a retention time (RT) difference greater than 2.5 minutes at all TFA levels (see FIG. 5). At 30 mM TFA, greater than 3.0 minutes of separation was achieved. TFA concentration had a more pronounced effect on the separation of VP1 and VP2. At an initial water content between 28 to 32 wt. % H₂O in the mobile phase, VP1 and VP2 had a RT difference greater than 0.70 minutes at all TFA levels (see FIG. 6). At 18 mM TFA, greater than 0.95 minutes of RT difference was measured.

### Example 3

### Elution Gradient and Flow Rate Optimization

A full factorial analysis was performed to evaluate elution gradients ranging from 0.400, 0.500, and 0.600 increase in wt. % H₂O/min and flow rates of the mobile phase ranging from 0.060 mL/min to 0.160 mL/min (Table 3). With a starting 28 wt. % H₂O in the mobile phase, a TFA concentration of 20 mM in the mobile phase, and a column temperature of 30° C., gradients of 0.400 wt. % increase in H₂O/min, 0.500 wt. % increase in H₂O/min, and 0.600 wt. % increase in H₂O/min were evaluated at flow rates of 0.060 mL/min, 0.080 mL/min, 0.100 mL/min, 0.120 mL/min, 0.140 mL/min, and 0.160 mL/min.

TABLE 3
Flow Rate and Gradient Optimization with Starting
H₂O conc. in the mobile phase of 28 wt. %

| | Elution gradient (wt. % increase of H₂O/min) | | |
|---|---|---|---|
| Flow (mL/min) | 0.400 | 0.500 | 0.600 |
| 0.060 | x | x | x |
| 0.080 | x | x | x |
| 0.100 | x | x | x |
| 0.120 | x | x | x |
| 0.140 | x | x | x |
| 0.160 | x | x | x |
| | Final H₂O (wt. %) in the mobile phase | | |
| | 52 | 58 | 64 |

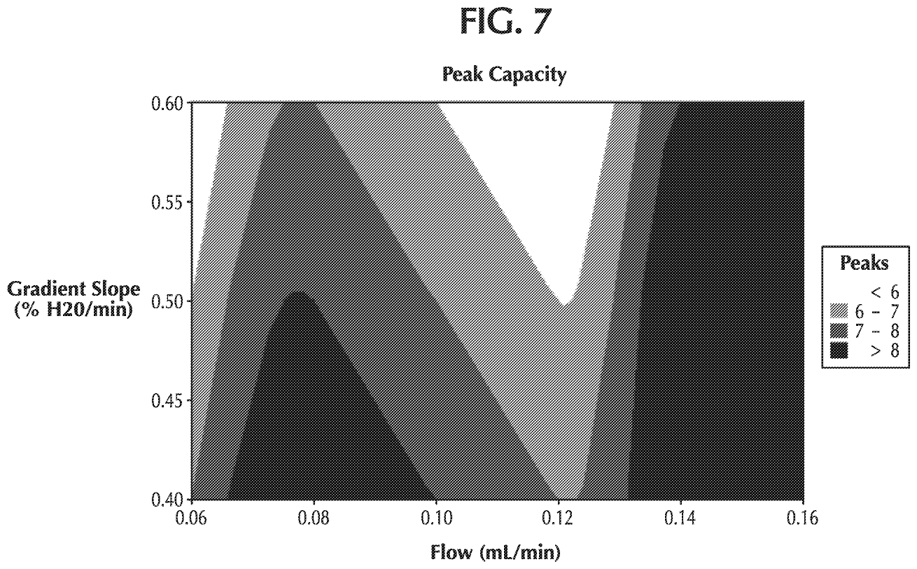

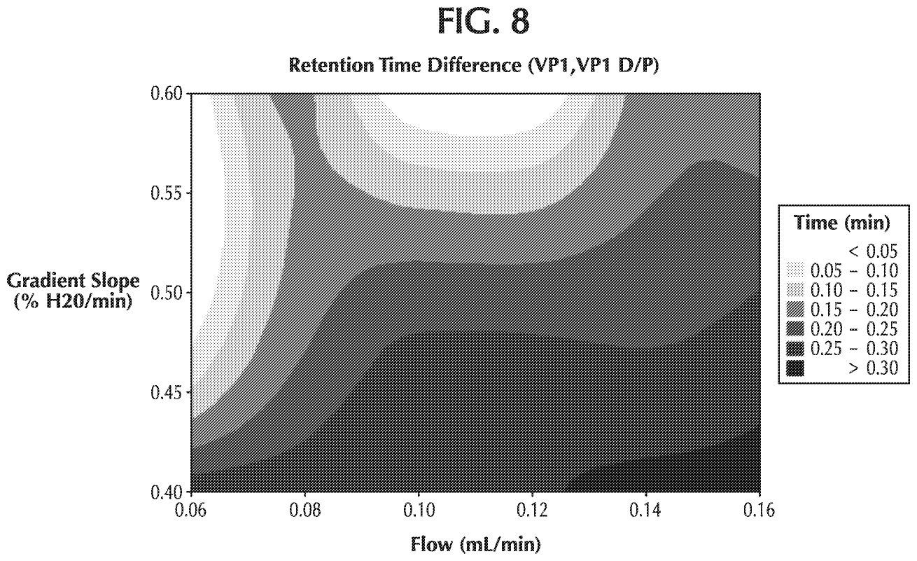

Peak capacity was the highest at flow rates greater than 0.140 mL/min at all elution gradients (see FIG. 7), with greater than 8 peaks detected. To determine the optimal gradient for AAV9, the retention time difference between VP1 and an adjacent minor peak was measured. The shallowest gradient, 0.400 wt. % H₂O/min, gave the largest retention time difference, greater than 0.30 minutes (see FIG. 8). The minor peak was later to be confirmed to be a mixture of deamidated and phosphorylated VP1 (VP1_{D/P} or VP1d, VP1p).

### Example 4

### Column Temperature

The effect of column temperature on separating major and minor species was investigated. VP1_{D/P} is a minor species that elutes between VP1 and VP2. The retention time difference of VP1_{D/P} to VP1 and VP2 was measured at 25, 30, 35, and 40° C. (Table 4). The samples were run with a starting 28 wt. % H₂O in the mobile phase, a TFA concentration of 20 mM in the mobile phase, a gradient slope of 0.400 wt. % H2O/min, and a Flow Rate of 0.140 mL/min at the specified column temperatures.

TABLE 4
Temperature Optimization
Column Temperature (° C.)
25
30
35
40

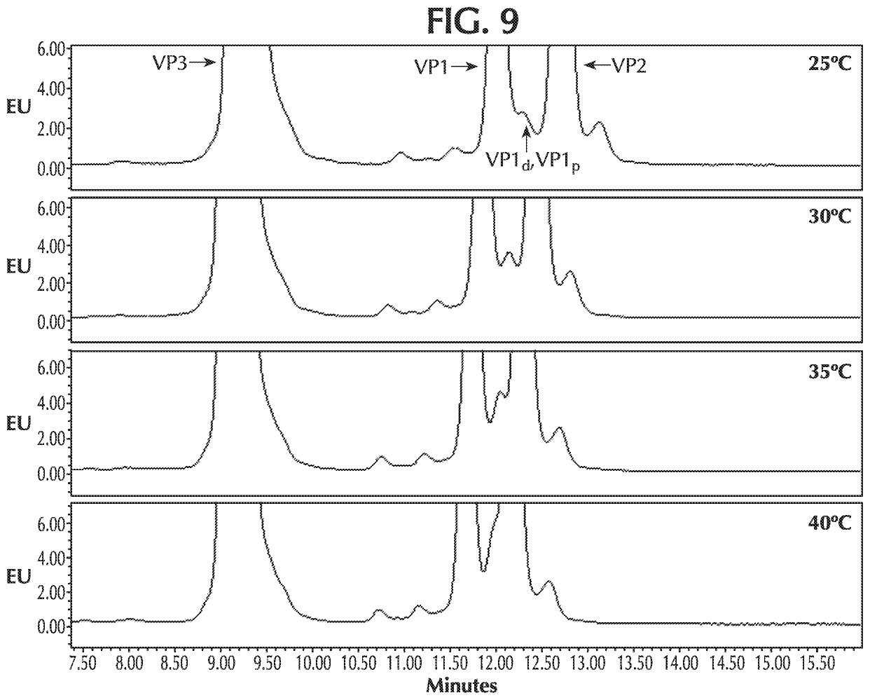

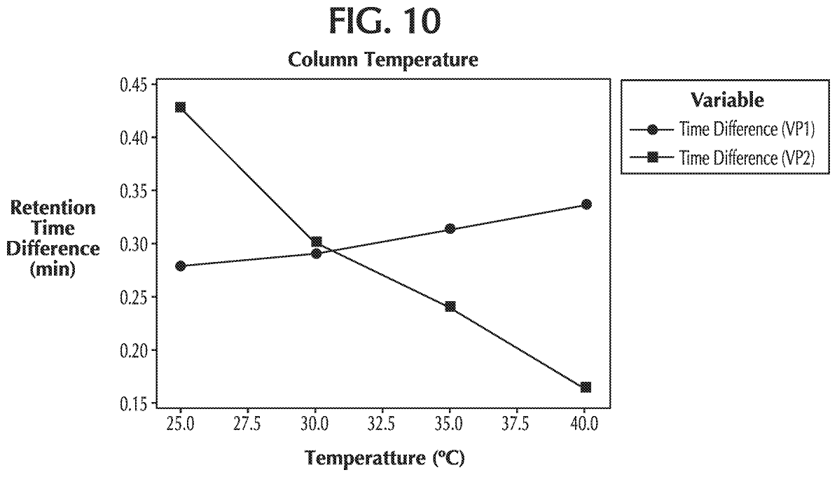

With increasing temperature, the retention time difference directly increases between VP1_{D/P} and VP1. In contrast, the retention time between VP1_{D/P} and VP2 decreases. (see FIG. 9). When retention time difference of VP1_{D/P} is plotted for VP1 and VP2 (see FIG. 10), the corresponding lines intersect at 30.6° C., indicating the optimal temperature for resolving VP1_{D/P} from both VP1 and VP2.

### Example 5

### Column Loading Under Optimized Conditions

From the previous experiments, the optimal conditions for separating AAV9 were determined and are set forth below in Table 5.

TABLE 5
Optimized HILIC Conditions for AAV9

| | |
|---|---|
| Starting H₂O (wt. %) of Mobile Phase | 28 |
| TFA (mM) | 20 |
| Gradient Slope (wt. % H₂O/min) | 0.400 |
| Flow Rate (mL/min) | 0.140 |
| Column Temperature (° C.) | 30.6 |
| Run Time (min) | 25 |

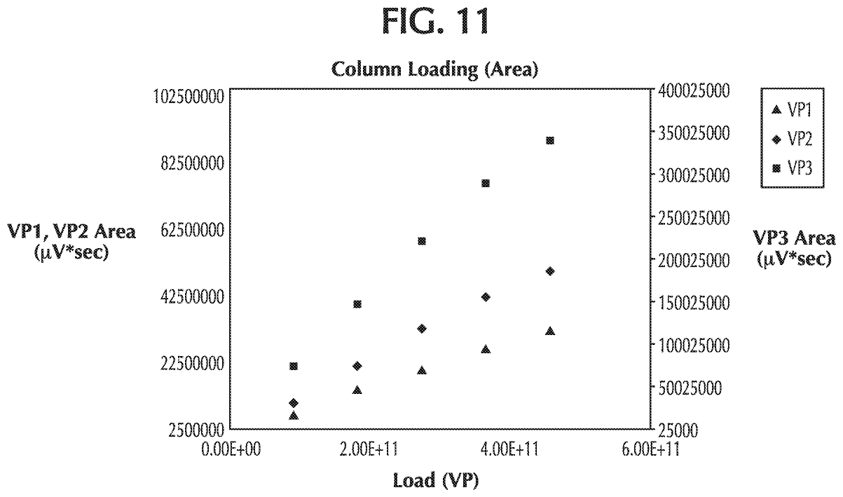

A five-point standard curve, with column loads ranging from 9.15×10¹⁰ to 4.58×10¹¹ vp/mL, was produced. Each level was injected in duplicate and the average was plotted versus column load (see FIG. 11). The calculated slopes, intercepts, and R 2 are tabulated in Table 6.

TABLE 6

| | | |
|---|---|---|
| Load VP | Area (μV*sec) | Difference (%) |

VP1

| | | |
|---|---|---|
| 9 × 10¹⁰ | 6720559 | 0.8 |
| 2 × 10¹¹ | 14403378 | 1.2 |
| 3 × 10¹¹ | 20421872 | 0.2 |
| 4 × 10¹¹ | 26834388 | 0.9 |
| 5 × 10¹¹ | 31785572 | 5.7 |
| Slope | 6.8 × 10⁻⁵ | |
| R² | 0.995 | |
| Intercept | 1264842.8 | |

VP2

| | | |
|---|---|---|
| 9 × 10¹⁰ | 10340190 | 0.4 |
| 2 × 10¹¹ | 21531429 | 0.6 |
| 3 × 10¹¹ | 32500892 | 0.5 |
| 4 × 10¹¹ | 42066659 | 0.3 |
| 5 × 10¹¹ | 49703854 | 5.3 |
| Slope | 1.1 × 10⁻⁴ | |
| R² | 0.994 | |
| Intercept | 1449837.05 | |

VP3

| | | |
|---|---|---|
| 9 × 10¹⁰ | 73172275 | 2.0 |
| 2 × 10¹¹ | 145165775 | 1.2 |
| 3 × 10¹¹ | 218529892 | 0.3 |
| 4 × 10¹¹ | 287268852 | 0.4 |
| 5 × 10¹¹ | 336319283 | 5.4 |
| Slope | 7.3 × 10⁻⁴ | |
| R² | 0.995 | |
| Intercept | 11572087.1 | |

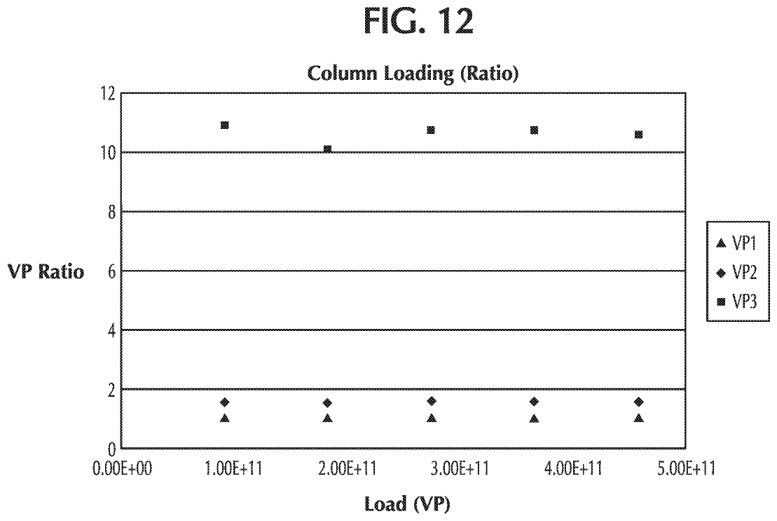

The capsid ratio as compared to VP1 was measured across all levels: VP1:1.0, VP2: 1.6±0.04, VP3: 10.6±0.31 (see FIG. 12 and Table 7).

TABLE 7
Capsid Ratios Observed at Various Column Loads

| | VP Ratio | | |
|---|---|---|---|
| Load VP | Ratio VP1 | Ratio VP2 | Ratio VP3 |
| 9. × 10¹⁰ | 1.0 | 1.5 | 10.9 |
| 2. × 10¹¹ | 1.0 | 1.5 | 10.1 |
| 3. × 10¹¹ | 1.0 | 1.6 | 10.7 |
| 4. × 10¹¹ | 1.0 | 1.6 | 10.7 |
| 5. × 10¹¹ | 1.0 | 1.6 | 10.6 |

### Example 6

### Additional Serotypes

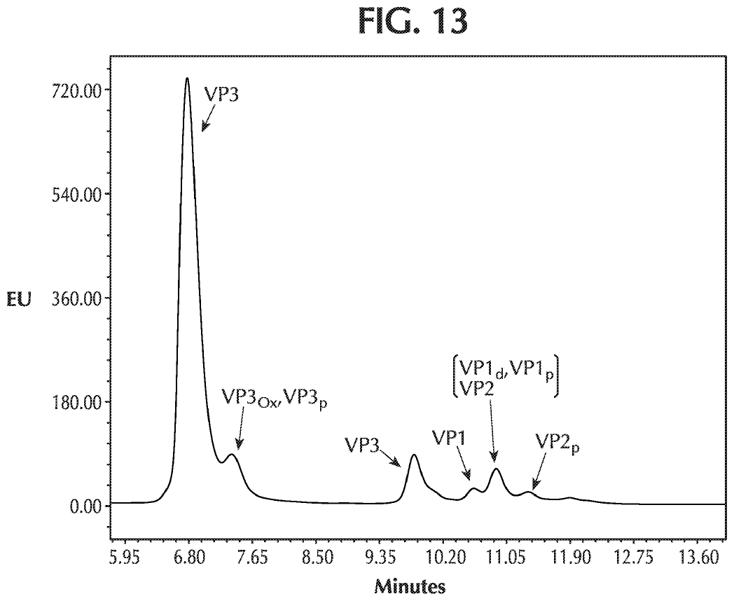

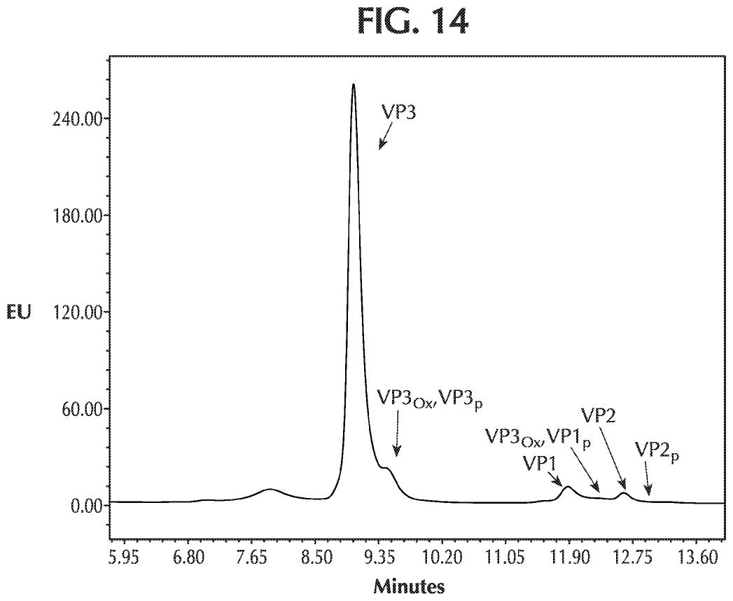

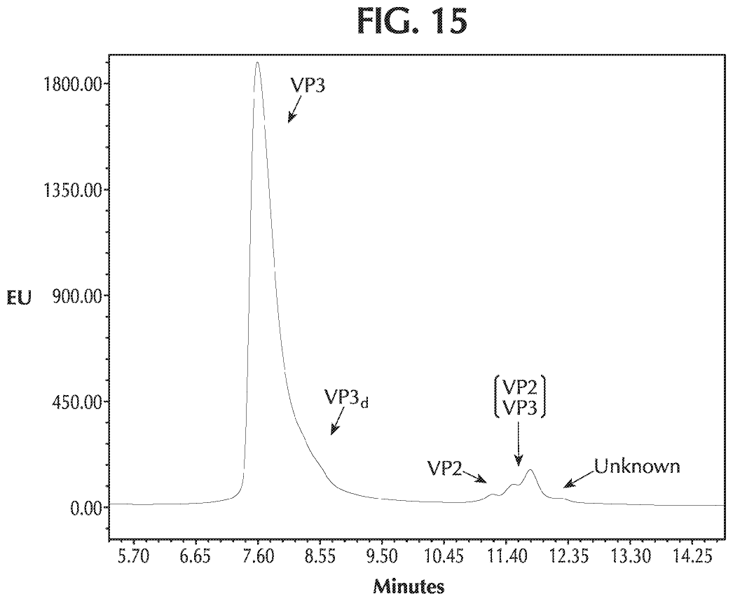

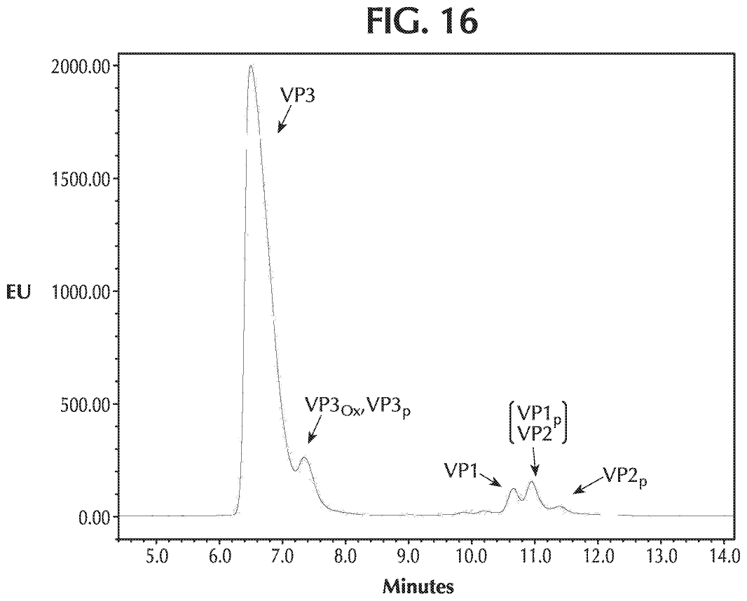

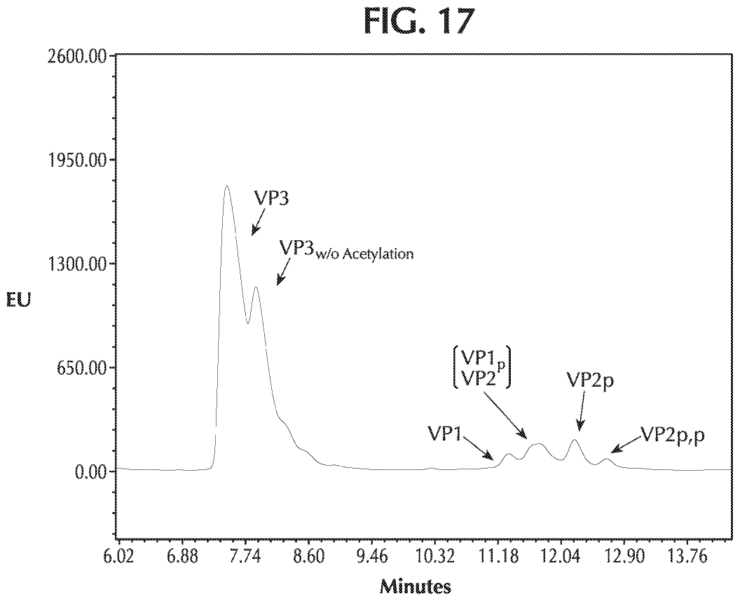

Additional serotypes (AAV1, AAV2, AAV5, AAV6, AAV8) were purchased and tested using the optimized conditions found for AAV9 as outlined in Table 5 above. Surprisingly, each serotype was able to be analyzed without changing the method (see FIGS. 13-17). The most abundant capsid protein, VP3, eluted first for all serotypes. Without interference from VP3, several minor species can be separated and identified by mass spectrometry. For AAV1, as can be seen in FIG. 13, two peaks for VP3 were observed. Both the peaks (e.g. proteins eluting at those peaks) had VP3 mass. However, the retention times of the two VP3 proteins were different, might be due to different conformation, or different degree of isomerization, such as aspartic acid to isoaspartic acid isomerization.

### Example 7

### Mass Spectrometry Identification of Modifications

Observed masses from serotypes were compared to theoretical capsid protein masses using the optimized conditions as outlined in Table 5 above. Results demonstrate the ability of the HILIC method to separate individual capsid proteins, in addition to product heterogeneity. Specifically, evidence of N-terminal processing, acetylation, phosphorylation, deamidation, and oxidation were observed based from the original intact molecular mass. These modifications are consistent with historical observations and could be confirmed by further LC-MS/MS studies in the future. Additionally, unknown modifications of the capsid proteins were observed in some cases but were not identified at the time. A majority of the observed masses matched the theoretical masses within 50 ppm for minor and trace level species, and within 20 ppm for major species (see Table 8). However, it should be noted that analysis was complicated by low concentration and significant presence of salt adducts related to the specific material used for the study.

TABLE 8
Mass Spectrometry Identification of Modifications

| | Capsid | | Theoretical | Observed | |
|---|---|---|---|---|---|
| Serotype | Protein | Residue | Mass | Mass | Modification |
| AAV1 | | | | 66807.3 | |
| AAV1 | | | | 67235.1 | |
| AAV1 | VP1 | [2-736] | 81286.0 | 81285.7 | Des-Met Ac |
| AAV1 | VP1 | [2-736] | 81287.0 | 81287.4 | Des-Met Ac + d |
| AAV1 | VP1 | [2-736] | 81365.9 | 81366.3 | Des-Met Ac + P |
| AAV1 | VP2 | [139-736] | 66093.2 | 66093.0 | Des-Thr |
| AAV1 | VP2 | [139-736] | 66173.2 | 66172.8 | Des-Thr + P |
| AAV1 | VP3 | [204-736] | 59516.9 | 59516.6 | Des-Met Ac |
| AAV1 | VP3 | [204-736] | 59532.9 | 59532.9 | Des-Met Ac + Ox |
| AAV1 | VP3 | [204-736] | 59596.9 | 59596.3 | Des-Met Ac + P |
| AAV2 | | | | 66590.7 | |
| AAV2 | VP1 | [2-735] | 81855.4 | 81855.3 | Des-Met Ac |
| AAV2 | VP1 | [2-735] | 81871.4 | 81872.1 | Des-Met Ac + Ox |
| AAV2 | VP1 | [2-735] | 81935.3 | 81935.8 | Des-Met Ac + P |
| AAV2 | VP2 | [139-735] | 66488.3 | 66491.8 | Des-Thr |
| AAV2 | VP2 | [139-735] | 66568.3 | 66564.8 | Des-Thr + P |
| AAV2 | VP3 | [204-735] | 59974.1 | 59973.9 | Des-Met Ac |
| AAV2 | VP3 | [204-735] | 59990.1 | 59993.9 | Des-Met Ac + Ox |
| AAV2 | VP3 | [204-735] | 60054.1 | 60052.2 | Des-Met Ac + P |
| AAV2 | VP3 | [204-735] | | 60179.5 | |
| AAV5 | | | | 65306.1 | |
| AAV5 | VP1 | [2-724] | 80335.5 | 80336.7 | Des-Met Ac |
| AAV5 | VP2 | [138-724] | 65283.1 | 65283.4 | Des-Thr |
| AAV5 | VP2 | [138-724] | 65325.1 | 65324.5 | Des-Thr + Ac |
| AAV5 | VP2 | [138-724] | 65456.3 | 65456.1 | AcMet |
| AAV5 | VP3 | [194-724] | 59462.7 | 59463.2 | Des-Met Ac |
| AAV5 | VP3 | [194-724] | 59463.7 | 59463.8 | Des-Met Ac + d |
| AAV6 | | | | 66810.6 | |
| AAV6 | VP1 | [2-736] | 81322.1 | 81322.4 | Des-Met Ac |
| AAV6 | VP1 | [2-736] | 81402.1 | 81402.4 | Des-Met Ac + P |
| AAV6 | VP2 | [139-736] | 66095.3 | 66095.4 | Des-Thr |
| AAV6 | VP2 | [139-736] | 66175.3 | 66173.8 | Des-Thr + P |
| AAV6 | VP3 | [204-736] | 59519.1 | 59518.8 | Des-Met Ac |
| AAV6 | VP3 | [204-736] | 59535.1 | 56538.3 | Des-Met Ac + Ox |
| AAV6 | VP3 | [204-736] | 59599.0 | 59598 | Des-Met Ac + P |
| AAV8 | | | | 67315.1 | |
| AAV8 | VP1 | [2-738] | 81667.2 | 81667.7 | Des-Met Ac |
| AAV8 | VP1 | [2-738] | 81747.1 | 81745.4 | Des-Met Ac + P |
| AAV8 | VP2 | [139-738] | 66518.5 | 66520 | Des-Thr |
| AAV8 | VP2 | [139-738] | 66598.4 | 66598.4 | Des-Thr + P |
| AAV8 | VP2 | [139-738] | 66678.4 | 66675.3 | Des-Thr + 2X P |
| AAV8 | VP3 | [205-738] | 59763.0 | 59762.6 | Des-Met |
| AAV8 | VP3 | [205-738] | 59805.0 | 59804.6 | Des-Met Ac |
| AAV9 | VP1 | [2-736] | 81290.8 | 81290.9 | Des-Met Ac |
| AAV9 | VP1 | [2-736] | 81291.8 | 81291.1 | Des-Met Ac + d |
| AAV9 | VP1 | [2-736] | 81370.8 | 81370.9 | Des-Met Ac + P |
| AAV9 | VP2 | [139-736] | 66210.1 | 66210.3 | Des-Thr |
| AAV9 | VP2 | [139-736] | 66211.1 | 66211.3 | Des-Thr + d |
| AAV9 | VP2 | [139-736] | 66226.1 | 66226 | Des-Thr + Ox |
| AAV9 | VP2 | [139-736] | 66290.1 | 66288.6 | Des-Thr + P |
| AAV9 | VP3 | [206-736] | 59733.0 | 59732.8 | Des-Met Ac |
| AAV9 | VP3 | [206-736] | 59734.0 | 59733.2 | Des-Met Ac + d |
| AAV9 | VP3 | [206-736] | 59812.9 | 59808.7 | Des-Met Ac + P |

For all figures it can be assumed that VP3 and VP1 is des-met with acetylation and that VP2 is des-thr. However, VP3 and VP1 can exist as des-met without acetylation at trace levels. Furthermore, in some instances when using a baculovirus manufacturing, the VP1 initiation codon may be leucine. In this case the VP1 is observed as des-leu with acetylation. While VP2 is observed primarily as des-thr, in some instances it can be detected with processed N-terminal residues (des-thr and ala, des-thr, ala, and pro [T, TA, and TAP missing from n-terminus]) or with acetylated methionine at the protein N-term.

The foregoing written specification is considered to be sufficient to enable one skilled in the art to practice the disclosure. The foregoing description and Examples detail certain exemplary embodiments of the disclosure. It will be appreciated, however, that no matter how detailed the foregoing may appear in text, the disclosure may be practiced in many ways and the disclosure should be construed in accordance with the appended claims and any equivalents thereof.

All references cited herein, including patents, patent applications, papers, text books, and the like, and the references cited therein, to the extent that they are not already, are hereby incorporated herein by reference in their entirety.

1. A method for separating proteins of a capsid of an adeno-associated virus (AAV) or recombinant adeno-associated virus (rAAV) particle, the method comprising:
a) loading the AAV particle onto a hydrophilic interaction liquid chromatography (HILIC) column;
eluting the HILIC column with a mobile phase comprising a 99.9 vol. % or more of a mixture of water, acetonitrile, and trifluoroacetic acid (TFA) to obtain an eluent, wherein the starting water concentration in the mobile phase is about 28 volume percent, the water concentration of the mobile phase is increased at a rate about 0.4 volume percentage per minute, TFA concentration in the mobile phase is about 20 mM, the mobile phase has a column flow rate of about 0.14 mL/min,
the HILIC column comprises a stationary phase comprising amide functional groups and the HILIC column temperature during elution of the mobile phase is 25° C. to 40° C.

2. The method of claim 1, further comprising performing mass spectrometry on at least a portion of the eluent and determining masses of one or more proteins in the eluent by mass spectrometry.

3. The method of claim 1 wherein the AAV or rAAV particle is loaded to the HILIC column by direct injection.

4. The method of claim 3 wherein the AAV or rAAV particle is loaded onto the HILIC column by direct injection of a neat sample of the AAV or rAAV particle.

5. The method of claim 3 wherein the AAV particle serotype is selected from the group consisting of AAV1, AAV2, AAV3, AAV3A, AAV3B, AAV4, AAV5, AAV6, AAV7, AAV8, AAV9, AAV10, AAVrh10, AAVrh74, AAV12, AAV2i8, NP4, NP22, NP66, AAVDJ, AAVDJ/8, AAVDJ/9, AAVLK03, AAV1.1, AAV2.5, AAV6.1, AAV6.3.1, AAV9.45, RHM4-1 (SEQ ID NO:5 of WO 2015/013313), RHM15-1, RHM15-2, RHM15-3/RHM15-5, RHM15-4, RHM15-6, AAV hu.26, AAV1.1, AAV2.5, AAV6.1, AAV6.3.1, AAV9,45, AAV2i8, AAV29G, AAV2,8G9, AVV-LK03, AAV2-TT, AAV2-TT-S312N, AAV3B-S312N, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14 and AAVHSC15.

6.-8. (canceled)

9. The method of claim 5 wherein intact AAV or rAAV particle is loaded to the HILIC.

10. The method of claim 5 wherein amounts of one or more proteins in the eluent is measured.

11. The method of claim 10 wherein the one or more proteins is selected from VP1, VP2, VP3, one or more post translation modification (PTM)s of VP1, one or more PTMs of VP2, and one or more PTMs of VP3, or any combination thereof.

12. The method of claim 11 wherein PTMs of VP1, VP2, and/or VP3 comprises independently acetylation, phosphorylation, deamidation, and/or oxidation thereof, or any combination thereof.

13. The method of claim 11 wherein masses and/or amounts of the one or more proteins is indicative of serotype of the AAV capsid.

14. The method of claim 11 wherein masses and/or amounts of the one or more proteins is indicative of heterogeneity of the AAV capsid.

15. The method of claim 11 wherein the AAV particle is AAV9 or rAAV9, and the one or more proteins is selected from a) VP1, b) des-methionine and acetylated VP1, c) des-methionine, acetylated, and deamidated VP1, d) des-methionine, acetylated, and phosphorylated VP1, e) VP2, f) des-threonine VP2, g) des-threonine, and deamidated VP2, h) des-threonine, and oxidized VP2, i) des-threonine and phosphorylated VP2, j) VP3, k) des-methionine and acetylated VP3, l) des-methionine, acetylated, and deamidated VP3, and m) des-methionine, acetylated and phosphorylated VP3, or any combination thereof.

16. The method of claim 11 wherein the AAV particle is AAV1, and the one or more proteins is selected from a) VP1, b) des-methionine and acetylated VP1, c) des-methionine, acetylated, and deamidated VP1, d) des-methionine, acetylated, and phosphorylated VP1, e) VP2, f) des-threonine VP2, g) des-threonine and phosphorylated VP2, h) VP3, i) des-methionine and acetylated VP3, j) des-methionine, acetylated, and oxidized VP3, and k) des-methionine, acetylated, and phosphorylated VP3, or any combination thereof.

17. The method of claim 11 wherein the AAV particle is AAV2, and the one or more proteins is selected from a) VP1, b) des-methionine and acetylated VP1, c) des-methionine, acetylated, and oxidized VP1, d) des-methionine, acetylated, and phosphorylated VP1, e) VP2, f) des-threonine VP2, g) des-threonine and phosphorylated VP2, h) VP3, i) des-methionine and acetylated VP3, j) des-methionine, acetylated, and oxidized VP3, and k) des-methionine, acetylated, and phosphorylated VP3, or any combination thereof.

18. The method of claim 11 wherein the AAV particle is AAV5, and the one or more proteins is selected from a) VP1, b) des-methionine and acetylated VP1, c) VP2, d) des-threonine VP2, e) des-threonine, and acetylated VP2, f) acetylated N-terminal methionine VP2, g) VP3, h) des-methionine and acetylated VP3, and i) des-methionine, acetylated, and deamidated VP3, or any combination thereof.

19. The method of claim 11 wherein the AAV particle is AAV6, and the one or more proteins is selected from a) VP1, b) des-methionine and acetylated VP1, c) des-methionine, acetylated, and oxidized VP1, d) des-methionine, acetylated, and phosphorylated VP1, e) VP2, f) des-threonine VP2, g) des-threonine and phosphorylated VP2, h) VP3, i) des-methionine and acetylated VP3, j) des-methionine, acetylated, and oxidized VP3 and k) des-methionine, acetylated, and phosphorylated VP3, or any combination thereof.

20. The method of claim 11 wherein the AAV particle is AAV8, and the one or more proteins is selected from a) VP1, b) des-methionine and acetylated VP1, c) des-methionine, acetylated, and oxidized VP1, d) des-methionine, acetylated, and phosphorylated VP1, e) VP2, f) des-threonine VP2, g) des-threonine and phosphorylated VP2, h) des-threonine and phosphorylated VP2, and d) des-threonine, and bisphosphorylated VP2, i) VP3, j) des-methionine and acetylated VP3, k) des-methionine, acetylated, and oxidized VP3, l) des-methionine, and m) des-methionine, acetylated, and phosphorylated VP3, or any combination thereof.

21. The method of claim 20 wherein the intact AAV or rAAV capsid is a null capsid.

22. A method for separating proteins of a capsid of an adeno-associated virus (AAV) or recombinant adeno-associated virus (rAAV) particle, the method comprising:
a) loading the AAV particle onto a hydrophilic interaction liquid chromatography (HILIC) column wherein the HILIC column comprises a stationary phase comprising amide functional groups;
eluting the HILIC column with a mobile phase comprising a 99.9 vol. % or more of a mixture of water, acetonitrile, and trifluoroacetic acid (TFA) to obtain an eluent, wherein the starting water concentration in the mobile phase is wherein the starting water concentration in the mobile phase is about 28 volume percent, the water concentration of the mobile phase is increased at a rate about 0.4 volume percentage per minute, TFA concentration in the mobile phase is about 20 mM, the mobile phase has a column flow rate of about 0.14 mL/min;
b) performing mass spectrometry on at least a portion of the eluent and determining masses of one or more proteins in the eluant by mass spectrometry; and
c) comparing the masses of the one or more capsid proteins determined in step c) with their expected theoretical mass.

23. The method of claim 22 wherein the AAV or rAAV particle is loaded onto the HILIC column by direct injection of a neat sample of the AAV or rAAV particle.

24. The method of claim 23 wherein the AAV particle serotype is selected from the group consisting of AAV1, AAV2, AAV3, AAV3A, AAV3B, AAV4, AAV5, AAV6, AAV7, AAV8, AAV9, AAV10, AAVrh10, AAVrh74, AAV12, AAV2i8, NP4, NP22, NP66, AAVDJ, AAVDJ/8, AAVDJ/9, AAVLK03, AAV1.1, AAV2.5, AAV6.1, AAV6.3.1, AAV9.45, RHM4-1 (SEQ ID NO:5 of WO 2015/013313), RHM15-1, RHM15-2, RHM15-3/RHM15-5, RHM15-4, RHM15-6, AAV hu.26, AAV1.1, AAV2.5, AAV6.1, AAV6.3.1, AAV9,45, AAV2i8, AAV29G, AAV2,8G9, AVV-LK03, AAV2-TT, AAV2-TT-S312N, AAV3B-S312N, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14 and AAVHSC15.

25. (canceled)

26. The method of claim 23 wherein intact AAV or rAAV particle is loaded to the HILIC.

27. The method of claim 26 wherein the intact AAV or rAAV capsid encapsidates a nucleic acid sequence.

28. The method of claim 24 wherein amounts of one or more proteins in the eluent is measured.

29. The method of claim 28 wherein the one or more proteins is selected from VP1, VP2, VP3, one or more post translation modification (PTM)s of VP1, one or more PTMs of VP2, and one or more PTMs of VP3, or any combination thereof.

30. The method of claim 29 wherein masses and/or amounts of the one or more proteins is indicative of serotype of the AAV capsid.

31. The method of claim 30 wherein wherein masses and/or amounts of the one or more proteins is indicative of heterogeneity of the AAV capsid and the heterogeneity comprises oxidized capsids, phosphorylated capsids, acetylated capsids, or truncated capsids, or any combination thereof.

32. The method of claim 31 wherein the AAV particle is AAV9 or rAAV9, and the one or more proteins is selected from a) VP1, b) des-methionine and acetylated VP1, c) des-methionine, acetylated, and deamidated VP1, d) des-methionine, acetylated, and phosphorylated VP1, e) VP2, f) des-threonine VP2, g) des-threonine, and deamidated VP2, h) des-threonine, and oxidized VP2, i) des-threonine and phosphorylated VP2, j) VP3, k) des-methionine and acetylated VP3, l) des-methionine, acetylated, and deamidated VP3, and m) des-methionine, acetylated and phosphorylated VP3, or any combination thereof.

33. The method of claim 31 wherein the AAV particle is AAV1, and the one or more proteins is selected from a) VP1, b) des-methionine and acetylated VP1, c) des-methionine, acetylated, and deamidated VP1, d) des-methionine, acetylated, and phosphorylated VP1, e) VP2, f) des-threonine VP2, g) des-threonine and phosphorylated VP2, h) VP3, i) des-methionine and acetylated VP3, j) des-methionine, acetylated, and oxidized VP3, and k) des-methionine, acetylated, and phosphorylated VP3, or any combination thereof.

34. The method of claim 31 wherein the AAV particle is AAV2, and the one or more proteins is selected from a) VP1, b) des-methionine and acetylated VP1, c) des-methionine, acetylated, and oxidized VP1, d) des-methionine, acetylated, and phosphorylated VP1, e) VP2, f) des-threonine VP2, g) des-threonine and phosphorylated VP2, h) VP3, i) des-methionine and acetylated VP3, j) des-methionine, acetylated, and oxidized VP3, and k) des-methionine, acetylated, and phosphorylated VP3, or any combination thereof.

35. The method of claim 31 wherein the AAV particle is AAV5, and the one or more proteins is selected from a) VP1, b) des-methionine and acetylated VP1, c) VP2, d) des-threonine VP2, e) des-threonine, and acetylated VP2, f) acetylated N-terminal methionine VP2, g) VP3, h) des-methionine and acetylated VP3, and i) des-methionine, acetylated, and deamidated VP3, or any combination thereof.

36. The method of claim 31 wherein the AAV particle is AAV6, and the one or more proteins is selected from a) VP1, b) des-methionine and acetylated VP1, c) des-methionine, acetylated, and oxidized VP1, d) des-methionine, acetylated, and phosphorylated VP1, e) VP2, f) des-threonine VP2, g) des-threonine and phosphorylated VP2, h) VP3, i) des-methionine and acetylated VP3, j) des-methionine, acetylated, and oxidized VP3 and k) des-methionine, acetylated, and phosphorylated VP3, or any combination thereof.

37. The method of claim 31 wherein the AAV particle is AAV8, and the one or more proteins is selected from a) VP1, b) des-methionine and acetylated VP1, c) des-methionine, acetylated, and oxidized VP1, d) des-methionine, acetylated, and phosphorylated VP1, e) VP2, f) des-threonine VP2, g) des-threonine and phosphorylated VP2, h) des-threonine and phosphorylated VP2, and d) des-threonine, and bisphosphorylated VP2, i) VP3, j) des-methionine and acetylated VP3, k) des-methionine, acetylated, and oxidized VP3, l) des-methionine, and m) des-methionine, acetylated, and phosphorylated VP3, or any combination thereof.
